(19) United States
(12) Patent Application Publication
(10) Pub. No.: US 2010/0197028 A1
(43) Pub. Date: Aug. 5, 2010

(54) METABOLIC MARKERS OF DIABETIC CONDITIONS AND METHODS OF USE THEREOF

(75) Inventors: Steven M. Watkins, Sacramento, CA (US); Michelle M. Wiest, knights Landing, CA (US)

Correspondence Address: Marshall, Gerstein & Borun LLP (TETHYS), 233 South Wacker Drive, 6300 Willis Tower, Chicago, IL, 60606, US

(73) Assignee: Tethys Bioscience, Inc., Emeryville, CA (US)

(21) Appl. No.: 12/528,065

(22) PCT Filed: Feb. 22, 2008
(86) PCT No.: PCT/US08/02357

§ 371 Date: Apr. 6, 2010

Related U.S. Application Data
(60) Provisional application No. 60/902,976, filed on Feb. 22, 2007.
(60) Provisional application No. 60/931,766, filed on May 24, 2007.
(60) Provisional application No. 61/021,853, filed on Jan. 17, 2008.

(51) Int. Cl. G01N 33/72; G01N 33/92

(52) U.S. Cl. 436/67; 436/71

(57) ABSTRACT

Novel methods for assessing the state of a diabetic condition of a subject are described, comprising determining the amount of a metabolite in a sample from a body fluid or tissue of the subject. The methods may be used, for example, in diagnosing and monitoring insulin resistance, prediabetes, or the response to a drug which alters a diabetic condition.

### CROSS-REFERENCE TO RELATED APPLICATIONS

This application claims the priority benefit of U.S. Provisional Application No. 60/902,976, filed Feb. 22, 2007, U.S. Provisional Application No. 60/931,766, filed May 24, 2007, and U.S. Provisional Application No. 61/021,853, filed Jan. 17, 2008, each of which is hereby incorporated by reference herein in its entirety.

### BACKGROUND OF THE INVENTION

Fifteen million people in the United States have type 2 diabetes. In both human and economic terms, diabetes is one of the most costly diseases in the nation today. The cost of medical care and services to treat diabetes is estimated to have been $91.8 billion in 2002. Another $40.2 billion of lost productivity, disability and premature death is also attributable to the disease. One million new cases are diagnosed each year, and many people do not learn they have the disease until they develop one of its life-threatening complications, which include heart disease, stroke and kidney disease.

Diabetes has been attributed to both genetic and lifestyle factors, including obesity, age, sedentary lifestyle, hypertension, and use of drugs that block insulin action or antagonize insulin action. As a result, in the absence of a predictive diagnostic, no single factor can be used to accurately assess an individual's propensity for developing the disease. Type 2 diabetes is currently diagnosed by measuring fasted plasma glucose, 2-hour plasma glucose or random plasma glucose (if symptoms are present). Persons with early-stage type 2 diabetes are usually asymptomatic and may not realize they are ill; they may live for many years with uncontrolled diabetes before symptoms ever occur. When they do occur, those symptoms are often related to a life-threatening complication. Early treatment of diabetes can delay or prevent complications from developing.

Treatment for prediabetes can slow or reverse the disease in some individuals, particularly in early stage disease. Lifestyle intervention or treatment with metformin in persons at high risk can reduce the incidence of diabetes by 58% and 31% respectively [1]. Hence, a plasma-based method to monitor early stage disease progression, and determine efficacy of treatment would greatly improve disease treatment.

### Type 2 Diabetes and Lipid Metabolism

More than one mechanism for the development of type 2 diabetes exists [2, 3]. While all of the genetic causes and environmental factors involved in development of insulin resistance are unknown, impaired lipid metabolism has been shown to play an important role in the development of type 2 diabetes. Increased fasting plasma fatty acids are correlated with the development of obesity and insulin resistance in many populations and are an independent predictor of the development of type 2 diabetes [4, 5].

One hypothesis for the development of increased plasma fatty acids and insulin resistance starts with the adipose tissue [6-8]. Enlarged adipocytes release inflammatory cytokines into the plasma which feed back to alter the adipose' and other tissue's response to insulin [9, 10]. As the adipocytes become insulin resistant, they are unable to suppress lipolysis in response to insulin. These adipocytes are also unable to store additional fat, consequently reducing the uptake of fatty acids after a meal, resulting in excess fatty acids in the plasma. The overwhelming amount of fatty acids released by adipose tissue chronically increases plasma levels and diverts lipid into other tissues including liver, muscle, and pancreas.

In the liver, the increased fatty acids stimulate gluconeogenesis and glucose output from the liver [11]. Chronic hyperinsulinemia and high plasma glucose concentrations stimulate liver de novo production of fatty acids [12, 13]. While the actual amount of fatty acids produced de novo is small, the conditions that increase fatty acid production also decrease liver fatty acid oxidation. This results in higher triglyceride esterification rates and increased availability of triglyceride for very low density lipoprotein synthesis and secretion. Along with the additional available substrate, decreased hepatocyte responsiveness to insulin may also increase release of very low density lipoprotein [14, 15]. The additional lipoprotein lipid released from the liver becomes substrate for lipase activity and release of free fatty acids into the plasma creating a positive feedback loop.

In the muscle, increased free fatty acids and intramuscular lipid is strongly correlated with impaired glucose metabolism [16]. The muscle responds to chronically increased plasma fatty acids by decreasing glucose uptake thus increasing fasting and postprandial plasma glucose concentrations [17, 18]. Muscle tissue may also increase uptake and decrease oxidation of the fatty acids, resulting in increased intramuscular lipid [19-21]. The decreased oxidative capacity of the muscle is due to dysfunctional mitochondria, though whether this is caused by the insulin resistant state, or a cause of it, is unknown.

Peripheral insulin resistance can exist without the development of overt diabetes [22-25]. Development of type 2 diabetes occurs when the pancreatic β-cells fail to compensate for insulin resistance by increasing insulin output [26]. The progression to diabetes is accompanied by loss of pancreatic β-cells as well as an increase in the basal rate of insulin secretion by the remaining cells, and the inability of these cells to respond to glucose [27]. The loss of function and cell death is due to chronic exposure of β-cells to high levels of both fatty acids and glucose [28-30]. Similar to the muscle, β-cells exposed to high concentrations of fatty acids have decreased lipid oxidation and increased intracellular triglycerides.

Type 2 diabetes is a disease of lipid metabolism as well as glucose metabolism [31]. While there are multiple mechanisms for the development of insulin resistance and type 2 diabetes, alterations in lipid metabolism is a common theme. Even though there are differences between individuals and groups of individuals in exactly how lipid metabolism is altered, disordered lipid storage and metabolism occurs at very early stages of insulin resistance in all individuals with insulin resistance and could be considered a marker of the disease. By monitoring lipid metabolites and whole body lipid metabolism, it may be possible to define the alterations in lipids that occur with insulin resistance and type 2 diabetes, segregate groups of patients by their changed lipid metabolism, and predict who would respond to therapy. Some lipids have been identified which predict the development of insulin resistance or diagnosis of insulin sensitivity [32-37]. However, the combination of specific lipids which improve the prediction of insulin resistance or diagnosis of a diabetic condition has not been previously shown.

What is needed are better testing methods that can be used to classify, diagnose, and monitor patients with prediabetes and insulin resistance, and to identify patients at risk of developing diabetes.

### Peroxisomal Proliferator Activated Receptors

Peroxisomal proliferator activated receptors (PPARs) are a group of nuclear receptor isoforms that function as transcription factors in cellular metabolism [38]. The receptors are connected to the metabolism of a cell and cell differentiation. There are three types of PPARs each produced by a separate gene: alpha, delta and gamma. PPARalpha is expressed primarily in the liver, kidney, heart, muscle, and adipose tissues, but is expressed at lower levels in other tissues as well. PPARdelta is expressed throughout the body in most tissues but at higher levels in skin, adipose and brain. PPARgamma has three forms of the protein all expressed by the same gene. Gamma1 is expressed in all tissues. Gamma2 is expressed primarily in the adipose tissue. Gamma3 is expressed in adipose tissue, macrophages and intestine.

The function and specificity of each of the PPARs is determined by the shape of their ligand-binding domain as well as co-activators and co-repressors [39, 40]. The endogenous ligands for the PPARs are free fatty acids and eicosanoids. Several classes of drug compounds are exogenous ligands for the PPARs. Fibrate drugs, such as clofibrate and fenofibrate, are ligands for PPAR alpha and are used to treat cholesterol disorders. Thiazolidinediones (TZDs) are ligands for PPARgamma and are used to treat diabetic conditions and lipodystrophies. Ligands for PPARdelta are currently in development and are used to increase fatty acid oxidation and improve insulin sensitivity.

Agents which modify the functions of PPARs include agonists, partial agonists, antagonists, dual agonists, selective receptor modulators; and agents such as antibodies that activate or inhibit activation of these receptors. These may also include agents that affect genes under the transcriptional control of a PPAR including agents which alter the binding of co-activators and co-repressors of PPARs.

### Eicosanoids

Eicosanoids are synthesized from polyenoic fatty acids in response to different biological stimuli. A large part of the oxidized lipids present in biofluids and tissues is specifically biosynthesized from polyunsaturated fatty acids by action of acutely regulated enzyme(s). Further conversion of the resulting unstable peroxide, epoxide, or hydroperoxide derivative results in a range of compounds that often exert potent biological effects. In humans, the fatty acid arachidonic acid is considered the most important precursor of oxygenated derivatives, compounds commonly referred to as eicosanoids (derived from 20 carbon chain length fatty acids). Most of the primary oxygenation of arachidonic acid and other fatty acids in animal tissue is catalyzed by cyclooxygenases (prostaglandin endoperoxide synthases) and lipoxygenases [41], and leads to a number of oxygenated derivatives such as prostaglandin H2, leukotriene A4 and various fatty acid hydroperoxides. These compounds can be further modified by secondary enzymes including prostaglandin E, D, and F synthases, thromboxane A synthase, prostacyclin synthase, leukotriene A4 hydrolase and leukotriene C4 synthase to generate members of the prostaglandin, leukotriene, and thromboxane families [42]. P-450 monooxygenase activity can also lead to the formation of epoxy, hydroxy, and dihydroxy derivative, whereas non-enzymatic oxygenation of arachidonic acid and other polyunsaturated fatty acids can lead to the formation of the isoprostane group of compounds [43]. Taken together, the eicosanoids exert remarkably diverse biological effects from acute cellular processes such as aggregation of blood platelets in response to bleeding, immune cell recruitment to injury and infection, to physiological processes such as reproduction. Not surprisingly, their dysregulation has been shown to be involved in pathological processes such as inflammatory reactions, autoimmunity, cancer and atherosclerosis. Inflammation is a critical component of diabetes and is involved in the development of comorbidities. In addition, specific eicosanoids bind to, and activate, the PPARs. Alteration of eicosanoid production in diabetes is the basis for treatment of diabetes and other inflammatory conditions with polyunsaturated fatty acids.

### Acylcarnitines

Carnitine (L-3-hydroxytrimethylammoniobutanoate) is an endogenous small metabolite with several established roles in mammalian cellular metabolism. The best described of carnitines biochemical functions are those mediated via the reversible transfer of activated carboxylic acids (“acyl” moieties) from coenzyme A to carnitine. Acyl carnitines form the substrate for carnitine palmitoyl transferase 1 that transfers fatty acid equivalents from the cell's cytoplasm to the mitochondrial interior where they form the substrates for beta oxidation. In this way, the formation of acylcarnitines is critical for the mitochondrial oxidation of long-chain fatty acids. Genetic mutations in genes for various aspects of acylcarnitine formation and transport are the basis of a variety of inborn errors of metabolism many of which are diagnosed by the excess accumulation of acylcarnitines in blood (WO 2003/104802) [44]. Individuals with such defects suffer from various forms of insufficient metabolic energy. An additional function of carnitine is in the transport of metabolites from ketones and amino acid metabolism and formation of short-chain acylcarnitines protects the cell from potentially toxic acyl-CoA accretion [45]. Thus, cells contain both carnitine and various chain length acylcarnitines, with the acylcarnitines composed of a wide range of specific acylcarnitines (i.e., myristoylcarnitine). In the published literature, total carnitine refers to free carnitine plus all acylcarnitines. The concentration of total carnitine in the plasma is increased in fasting humans, however, levels are lowered by obesity and diabetes [46-48]. The plasma level of specific acylcarnitines, such as acetyl carnitine, is highly correlated with the plasma concentration of ketones [46, 47, 49, 50]. In muscle and myocardium, the distribution and concentration of acylcarnitine molecular species are altered by diabetes and ischemia [51]. The decreased levels of carnitine and acetyl carnitine in type 1 and type 2 diabetes has led to supplementation of these compounds to prevent or treat the disease (U.S. Pat. No. 4,362,719, U.S. Pat. No. 7,060,295).

All publications, patents, patent applications, internet sites, and accession numbers/database sequences (including both polynucleotide and polypeptide sequences) cited herein are hereby incorporated by reference herein in their entirety for all purposes to the same extent as if each individual publication, patent, patent application, interne site, or accession number/database sequence were specifically and individually indicated to be so incorporated by reference.

### BRIEF SUMMARY OF THE INVENTION

In one aspect, the invention provides a method for assessing a diabetic condition in a subject comprising measuring the level of one or more metabolite marker in a sample from a subject. In some embodiments, the one or more metabolite markers are selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, CE18:1n9, PC18:1n9, and PE16:0. In some embodiments, the method comprises a step of correlating the levels of the one or more markers with the presence, absence, risk of developing, progression, regression, and/or severity of the diabetic condition.

In another aspect, the invention provides a method of assessing the response of a subject having a diabetic condition to a treatment for the diabetic condition comprising measuring the level of one or more metabolite markers in a sample from the subject following administration of the treatment to the subject. In some embodiments, the one or more metabolite markers are selected from the group consisting of: the relative amount of 14:0 to total fatty acid content in TG; the relative amount of 14:0 to total fatty acid content in total lipids; the relative amount of 16:0 to total fatty acid content in PC; the relative amount of 16:0 to total fatty acid content in TG; the relative amount of 16:0 to total fatty acid content in total lipids; the relative amount of 16:1n7 to total fatty acid content in PC; the relative amount of 16:1n7 to total fatty acid content in CE; the relative amount of 16:1n7 to total fatty acid content in TG; the relative amount of 16:1n7 to total fatty acid content in FA; the relative amount of 16:1n7 to total fatty acid content in total lipids; the relative amount of 18:1n9 to total fatty acid content in PC; the relative amount of 18:1n9 to total fatty acid content in CE; the relative amount of 18:1n9 to total fatty acid content in total lipids; the relative amount of 20:3n9 to total fatty acid content in PC; the relative amount of 20:3n9 to total fatty acid content in CE; the relative amount of 20:3n9 to total fatty acid content in TG; the relative amount of 20:3n9 to total fatty acid content in total lipids; the relative amount of 20:3n6 to total fatty acid content in PC; the relative amount of 20:3n6 to total fatty acid content in CE; the relative amount of 20:3n6 to total fatty acid content in total lipids; the relative amount of 18:1n9 to total fatty acid content in FA; the relative amount of 22:6n3 to total fatty acid content in PC; the relative amount of 22:6n3 to total fatty acid content in CE; the relative amount of 22:6n3 to total fatty acid content in TG; the relative amount of 22:6n3 to total fatty acid content in total lipids; the relative amount of 18:0 to total fatty acid content in PC; the relative amount of 18:0 in total lipids to total fatty acid content; the relative amount of 18:2n6 to total fatty acid content in PC; the relative amount of 18:2n6 to total fatty acid content in CE; the relative amount of 18:2n6 to total fatty acid content in FA; the relative amount of 18:2n6 to total fatty acid content in TG; the relative amount of 18:2n6 to total fatty acid content in total lipids; the relative amount of 18:3n6 to total fatty acid content in PC; the relative amount of 18:3n6 to total fatty acid content in CE; the relative amount of 18:3n6 to total fatty acid content in TG; the relative amount of 18:3n6 to total fatty acid content in total lipids; the relative amount of 20:3n6 to total fatty acid content in PC; the relative amount of 20:3n6 to total fatty acid content in CE; and the relative amount of 20:3n6 to total fatty acid content in total lipids.

In an additional aspect, the invention further provides methods of assessing the level of a diabetic condition in a subject are provided, comprising determining the amount of a lipid metabolite in a sample from a body fluid of the subject. In one embodiment, the lipid metabolite is a fatty acid present in a lipid class. In one embodiment, the lipid class is selected from the group consisting of: free fatty acids, diglycerides, lysophosphatidylcholines, total fatty acids, triglycerides, cholesterol esters, phosphatidylcholine, and phosphatidylethanolamine. In one embodiment, the amount of the metabolite is the relative amount of a fatty acid to total fatty acid content in the lipids of one or more lipid classes in the sample. In one embodiment, the relative amount is selected from the group consisting of: (a) the relative amount of a fatty acid to total fatty acid content in triglycerides in the sample; (b) the relative amount of a fatty acid to total fatty acid content in free fatty acids in the sample; (c) the relative amount of a fatty acid to total fatty acid content in phosphatidylcholines in the sample; (d) the relative amount of a fatty acid to total fatty acid content in phosphatidylethanolamines in the sample; (e) the relative amount of a fatty acid to total fatty acid content in cholesterol esters in the sample; and (f) the relative amount of a fatty acid to total fatty acid content in all lipids in the sample. In one embodiment, the fatty acid is selected from the group consisting of: CE14.0, CE16.0, CE20.0, CE16:1n7, CE18.1n7, CE18.1n9, CE18.2n6, CE18.3n6, CE22:2n6, CE20.3n9, CE22.5n6, DG16:0, DG18.0, DG18.2n6, DG18.3n6, DG20:0, DG20.3n6, DG20.3n9, DG22.1n9, FA14.0, FA15.0, FA16.0, FA16.1n7, FA18.0, FA18.1n9, FA18.1n7, FA18.2n6, FA20.4n6, FA22.2n6, FA22.4n6, FA20.5n3, FA22.6n3, FA24.1n9, LY18.0, LY16.1n7, LY18:1n7, LY18.1n9, LY20.3n9, LY18.2n6, LY20:3n6, LY22:4n6, LY22:5n3, PC14.0, PC16.1n7, PC18.0, PC15.0, PC18.1n7, PC18.1n9, PC18.2n6, PC18.3n6, PC18.3n3, PC20.1n9, PC20:3n9, PC20:4n3, PC20.2n6, PC20.4n6, PC22.4n6, PC22.5n3, PCdm16.0, PCdm18.0, PCdm18.1n9, PCdm18:1n7, PE14.0, PE16.0, PE20.0, PE16.1n7, PE18.1n9, PE18:3n6, PE20.0, PE20.1n9, PE20:3n9, PE20:3n6, PE20.4n6, PE20.5n3, PEdm16.0, PEdm18.0, TG14.0, TG14.1n5, TG16.0, TG20.0, TG16.1n7, TG18.1n7, TG18.1n9, TG18.2n6, TG20.2n6, TG20.3n6, TG20.3n9, TG22.2n6, TG22:4n6, CETotal.LC, TGTotal.LC, DGTotal.LC, FSTotal.LC, AC6:0, AC16:0, AC14:0, AC8:0, AC10:0, AC3:0, AC12:0, L-Carnitine, and AC4:0.

In one embodiment, the sample is selected from the group consisting of blood, plasma, serum, isolated lipoprotein fraction, saliva, urine, lymph fluid, and cerebrospinal fluid. In another embodiment, the sample is selected from the group consisting of blood, plasma, serum, or isolated lipoprotein fraction. In one embodiment, the sample is lymph or cerebrospinal fluid.

Methods of assessing the diabetic condition of a subject may be used in diagnosing, monitoring, assessing the severity, and/or assessing the progression or regression of a diabetic condition, wherein the condition is selected from the group consisting of: diabetes, type 2 diabetes, insulin resistance, impaired glucose tolerance, impaired fasting glucose, prediabetes, metabolic syndrome, hepatic steatosis, insulin sensitivity, hyperinsulinemia, hepatic steatosis, muscle steatosis, hyperlipidemia, hypercholesterolemia.

In another aspect of the invention, methods of assessing the response to an intervention which modifies the action of a PPARgamma in a subject are provided, comprising determining the amount of a lipid metabolite in a sample from a body fluid of the subject. In one embodiment, the lipid metabolite is a fatty acid present in a lipid class. In one embodiment, the lipid class is selected from the group consisting of: free fatty acids, diglycerides, lysophosphatidylcholines, total fatty acids, triglycerides, cholesterol esters, phosphatidylcholine, and phosphatidylethanolamine. In one embodiment, the amount of the metabolite is the relative amount of a fatty acid to total fatty acid content in the lipids of one or more lipid classes in the sample. In one embodiment, the relative amount is selected from the group consisting of: (a) the relative amount of a fatty acid to total fatty acid content in triglycerides in the sample; (b) the relative amount of a fatty acid to total fatty acid content in free fatty acids in the sample; (c) the relative amount of a fatty acid to total fatty acid content in phosphatidylcholines in the sample; (d) the relative amount of a fatty acid to total fatty acid content in phosphatidylethanolamines in the sample; (e) the relative amount of a fatty acid to total fatty acid content in cholesterol esters in the sample; and (f) the relative amount of a fatty acid to total fatty acid content in all lipids in the sample. In one embodiment, the fatty acid is selected from the group consisting of: PC20:4n3, PC16:1n7, CE16:1n7, CE18:1n9, LY20:3n6, PC18:1n9, CE20:2n6, FA24:0, PE20:3n9, CE20:3n9, PC20:3n9, PE20:3n6, LY18:1n7, TG16:1n7, FA14:0, FA16:1n7, FA22:6n3, FA20:5n3, PC20:2n6, CETotal.LC, TG16:0, PC20:3n6, PE18:1n7, PE18:2n6, CE18:0, PE16:1n7, CE18:1n7, PE16:0, LY20:3n9, PC18:1n7, LY20:1n9, CE14:0, FA18:1n7, TG14:0, PC20:1n9, CE20:3n6, TG18:1n7, LY18:1n9, LY16:0, PC16:0, DGTotal.LC, DG16:0, DG18:0, LYTotal.LC, PETotal.LC, PC20:4n6, CE20:4n6, TG22:4n6, PC20:0, LY22:5n3, FA18:1n9, DG18:1n9, LY20:5n3, PC22:6n3, FATotal.LC, TG22:6n3, PE20:4n6, LY18:0, PC18:0, FA22:5n3, CE18:2n6, LY20:4n6, FA18:2n6, LY18:2n6, DG18:2n6, PC18:4n3, LY18:3n3, TG20:5n3, DG20:4n6, TG20:4n6, PC18:3n3, TG18:3n3, Pedm, TG18:4n3, TG18:2n6, PCdm16:0, PEdm18:0, PEdm18:1n9, PC14:0, TG22:0, TG18:3n6, CE16:0, SP18:0.

In one embodiment, the sample is selected from the group consisting of blood, plasma, serum, isolated lipoprotein fraction, saliva, urine, lymph fluid, and cerebrospinal fluid. In another embodiment, the sample is selected from the group consisting of blood, plasma, serum, or isolated lipoprotein fraction. In one embodiment, the sample is lymph or cerebrospinal fluid.

In another aspect of the invention, methods of assessing the response to an intervention which modifies the action of a PPARalpha in a subject are provided, comprising determining the amount of a lipid metabolite in a sample from a body fluid of the subject. In one embodiment, the lipid metabolite is a fatty acid present in a lipid class. In one embodiment, the lipid class is selected from the group consisting of: free fatty acids, diglycerides, lysophosphatidylcholines, total fatty acids, triglycerides, cholesterol esters, phosphatidylcholine, and phosphatidylethanolamine. In one embodiment, the amount of the metabolite is the relative amount of a fatty acid to total fatty acid content in the lipids of one or more lipid classes in the sample. In one embodiment, the relative amount is selected from the group consisting of: (a) the relative amount of a fatty acid to total fatty acid content in triglycerides in the sample; (b) the relative amount of a fatty acid to total fatty acid content in free fatty acids in the sample; (c) the relative amount of a fatty acid to total fatty acid content in phosphatidylcholines in the sample; (d) the relative amount of a fatty acid to total fatty acid content in phosphatidylethanolamines in the sample; (e) the relative amount of a fatty acid to total fatty acid content in cholesterol esters in the sample; and (f) the relative amount of a fatty acid to total fatty acid content in all lipids in the sample. In one embodiment, the fatty acid is selected from the group consisting of: CE16:1n7, CE18:1n9, CE18:3n6, CE20:3n9, CE20:4n6, DG14:0, DG14:1n5, DG15:0, DG16:0, DG18:0, DG20:4n6, DG22:6n3, DG24:0, FA14:1n5, FA15:0, FA16:0, FA18:0, FA20:0, FA22:0, FA22:1n9, FA24:0, FA24:1n9, LY16:0, LY18:3n6, LY20:4n3, PC16:0, PC16:1n7, PC18:1n9, PC18:3n6, PC18:4n3, PC20:2n6, PC20:3n6, PC20:3n9, PC20:4n3, PCdm16:0, PCdm18:1n7, PE16:1n7, PEdm16:0, PEdm18:1n7, TG15:0, TG16:0, TG16:1n7, TG20:3n9, TG20:4n6, TG22:4n6, TG22:5n6, TG24:0, TG18.3n6, TG18.4n3, CE18:2n6, CETotal.LC, DG18:1n7, DG18:1n9, DG18:2n6, DGTotal.LC, FA18:1n9, FA18:2n6, FA20:1n9, FATotal.LC, PC18:2n6, PC22:5n3, PE18:0, PE22:0, PE22:1n9, TG18:2n6, TG18:3n3, TGTotal.LC.

In one embodiment, the sample is selected from the group consisting of blood, plasma, serum, isolated lipoprotein fraction, saliva, urine, lymph fluid, and cerebrospinal fluid. In another embodiment, the sample is selected from the group consisting of blood, plasma, serum, or isolated lipoprotein fraction. In one embodiment, the sample is lymph or cerebrospinal fluid.

In another aspect of the invention, methods of assessing the response to an intervention which modifies the action of a PPARdelta in a subject are provided, comprising determining the amount of a lipid metabolite in a sample from a body fluid of the subject. In one embodiment, the lipid metabolite is a fatty acid present in a lipid class. In one embodiment, the lipid class is selected from the group consisting of: free fatty acids, diglycerides, lysophosphatidylcholines, total fatty acids, triglycerides, cholesterol esters, phosphatidylcholine, and phosphatidylethanolamine. In one embodiment, the amount of the metabolite is the relative amount of a fatty acid to total fatty acid content in the lipids of one or more lipid classes in the sample. In one embodiment, the relative amount is selected from the group consisting of: (a) the relative amount of a fatty acid to total fatty acid content in triglycerides in the sample; (b) the relative amount of a fatty acid to total fatty acid content in free fatty acids in the sample; (c) the relative amount of a fatty acid to total fatty acid content in phosphatidylcholines in the sample; (d) the relative amount of a fatty acid to total fatty acid content in phosphatidylethanolamines in the sample; (e) the relative amount of a fatty acid to total fatty acid content in cholesterol esters in the sample; and (f) the relative amount of a fatty acid to total fatty acid content in all lipids in the sample. In one embodiment, the fatty acid is selected from the group consisting of: CE16:1n7, CE18:1n9, CE18:3n6, CE20:3n9, DG14:0, DG15:0, DG16:0, DG16:1n7, FA14:0, FA14:1n5, FA15:0, FA18:0, FA20:0, FA20:4n6, FA22:0, FA22:2n6, FA22:5n6, FA24:1n9, LY16:1n7, LY18:1n9, LY18:3n6, LY20:3n9, PC16:1n7, PC18:1n9, PC18:3n3, PC18:3n6, PC20:2n6, PC20:3n9, PC20:4n3, PC20:5n3, PCdm16:0, PCdm18:1n9, PE16:1n7, PE18:1n7, PE20:3n9, TG14:0, TG14:1n5, TG16:0, TG16:1n7, TG18:3n6, TG18:4n3, TG20:3n9, TG20:4n6, TG22:4n6, TG24:1n9, L-carnitine and butyrobetaine, CE18:1n7, CE18:2n6, CE20:4n6, CE22:1n9, CETotal.LC, DG18:2n6, FA18:1n7, FA18:1n9, FA20:1n9, FA22:6n3, FATotal.LC, LY18:0, LY20:4n6, LY22:6n3, PC15:0, PC20:4n6, PC22:5n6, PC22:6n3, PE18:0, PE22:6n3, TG18:2n6, TG18:3n3, CE16:0, DG18:3n3, DG20:3n6, DGTotal.LC, FA18:2n6, FA20:2n6, FA20:3n6, PC18:2n6, PE20:2n6, PEdm18:0, PETotal.LC and TGTotal.LC.

In one embodiment, the sample is selected from the group consisting of blood, plasma, serum, isolated lipoprotein fraction, saliva, urine, lymph fluid, and cerebrospinal fluid. In another embodiment, the sample is selected from the group consisting of blood, plasma, serum, or isolated lipoprotein fraction. In one embodiment, the sample is lymph or cerebrospinal fluid.

Additional biomarkers and examinations may be used in the methods of diagnosing, monitoring, assessing severity, and assessing progression or regression of the diabetic condition or in assessing the response to an intervention which modifies the action of a PPAR. In one embodiment, the method further comprises: (c) determining the level of malonyl-CoA or malonyl carnitine in a body fluid or cellular sample from the subject; (d) determining the level of an acylcarnitine, free carnitine, or butyrobetaine in a body fluid or cellular sample from the subject; and/or (e) determining the level of a sterol or bile acid in a body fluid or cellular sample from the subject. In one embodiment, the acylcarnitine is an acylcarnitine in Table 2. In one embodiment, the sterol or bile acid is a sterol or bile acid in Table 3. In another embodiment, the method further comprises the step of determining the level of an eicosanoid in a body fluid or cellular sample from the subject. In one embodiment, the eicosanoid is an eicosanoid in Table 4. In another embodiment, the method further comprises the step of determining the level of a cytokine, chemokine, adipokine, leptin, TNF, or C-reactive protein in a body fluid or cellular sample from the subject. In one embodiment, the cytokine, chemokine, adipokine, leptin, TNF, IL-6, or C-reactive protein.

In still another aspect, the invention provides a method for assessing a diabetic condition in a subject comprising measuring the level of a first metabolite marker in a sample from a subject wherein the first metabolite marker is selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, CE18:1n9, PC18:1n9, and PE16:0. In some embodiments, the first metabolite marker is selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, and PE16:0. In some embodiments the amount of the lipid metabolite measured is an absolute amount (e.g. nMoles per gram of plasma or serum). In some embodiments, the amount of the lipid metabolite measured is a relative amount (e.g., the relative amount of one or more fatty acids to total fatty acid content in one or more lipid classes). In some embodiments, for markers in the CE, DG, FA, LY, PC, PE and TG lipid classes, the indicated fatty acid components or components are quantified as a proportion of total fatty acids within the indicated lipid class or classes (e.g., mole percent composition of the fatty acid or fatty acids in the one or more lipid classes). In some embodiments, for markers in the AC lipid class, the indicated fatty acid-carnitine esters are quantified in absolute terms (e.g. nMoles per gram of plasma or serum). In some embodiments, the level of the first metabolite marker is indicative of the presence, absence, or severity of the diabetic condition. In some embodiments, a second, third, fourth, fifth, sixth, seventh, eighth, ninth, and/or tenth metabolite marker selected from the group is also measured and the levels of the measured markers is indicative of the presence, absence, or degree of a diabetic condition. In some embodiments, the first (and/or second, third, fourth, fifth, sixth, seventh, eighth, ninth, and/or tenth) metabolite marker is selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, and CE18:2n6. In some embodiments, the first (and/or second, third, fourth, fifth, sixth, seventh, eighth, ninth, and/or tenth) metabolite marker is selected from the group consisting of AC6:0, AC16:0, AC14:0, AC8:0, AC10:0, AC12:0, TG14:0, FA16:1n7, FA18:1n9, CE16:1n7, PC16:1n7 and PC18:1n9. In some embodiments, the first (and/or second, third, fourth, fifth and/or sixth) metabolite marker is selected from the group consisting of AC6:0, AC8:0, AC10:0, TG14:0, FA16:1n7, and/or PC18:1n9. In some embodiments, the diabetic condition is impaired glucose tolerance, insulin resistance, insulin sensitivity, hepatic steatosis, non-alcoholic steatohepatitis (NASH), pediatric NASH, obesity, childhood obesity, metabolic syndrome, polycystic ovary disease, or gestational diabetes. In some embodiments, the diabetic condition is a prediabetic condition. In some embodiments, the diabetic condition is impaired glucose tolerance or is insulin resistance. In some embodiments, the sample is blood, plasma, serum, or isolated lipoprotein fraction. In some embodiments, measuring the level of the first metabolite marker includes chromatography, immunoassay, enzymatic assay, or mass spectroscopy. In some embodiments, the method of assessing the diabetic condition is a method of diagnosing, identifying, monitoring, and/or assessing the severity of the diabetic condition. In some embodiments the subject is being monitored for response to treatment for the diabetic condition (including, but not limited to, treatment with a PPARs-gamma agonist, PPARs-alpha agonist, and/or PPARs-delta agonist).

In a further aspect, the invention provides a method for assessing a diabetic condition in a subject comprising measuring the level or levels of a first, second, third, fourth, fifth, sixth, seventh, eighth, ninth, and/or tenth metabolite marker in a sample from a subject wherein the first, second, third, fourth, fifth, sixth, seventh, eighth, ninth, and/or tenth metabolite marker is selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, CE18:1n9, PC18:1n9, and PE16:0. In some embodiments, the level or levels of the first, second, third, fourth, fifth, sixth, seventh, eighth, ninth, and/or tenth metabolite marker selected from the group are indicative of the presence, absence, risk of developing, and/or degree (or severity) of a diabetic condition (either independently or in combination). In some embodiments, the method further comprises correlating the level of the first, second, third, fourth, fifth, sixth, seventh, eighth, ninth, and/or tenth metabolite markers with the presence, absence, risk of developing and/or degree (or severity) of the diabetic condition. In some embodiments, if a marker that is measured is AC6:0, PE20:4n6, AC16:0, AC14:0, FA22:2n6, AC8:0, AC10:0, AC3:0, CETotal:LC, AC12:0, TGTotal:LC, PC18:0, L-Carnitine, PE20:0, DGTotal:LC, AC4:0, TG18:1n9, PE20:4n6, PC20:4n6, CE14:0, CETotal:LC, FSTotal:LC, or PCdm, then the marker is positively correlated with the presence, risk of developing, or severity of the diabetic condition. In some embodiments, if a marker that is measured is TG14:0, PE16:1n7, PE20:0, PC18:2n6, DG18:0 and CE18:2n6, CE16:1n7, CE18:1n9, PC16:1n7, PC18:1n9, FA16:1n7, FA18:1n9, TG16:1n7, or PE16:0, then the marker is negatively correlated with the presence, risk of developing, or severity of the diabetic condition. In some embodiments, the first, second, third, fourth, fifth, sixth, seventh, eighth, ninth, and/or tenth metabolite marker are selected from the group consisting of: AC6:0, AC16:0, AC14:0, AC8:0, AC10:0, AC12:0, TG14:0, FA16:1n7, FA18:1n9, CE16:1n7, PC16:1n7 and PC18:1n9. In some embodiments the amount of the lipid metabolite measured is an absolute amount (e.g. nMoles per gram of plasma or serum). In some embodiments, the amount of the lipid metabolite measured is a relative amount (e.g., the relative amount of one or more fatty acids to total fatty acid content in one or more lipid classes). In some embodiments, for markers in the CE, DG, FA, LY, PC, PE and TG lipid classes, the indicated fatty acid components or components are quantified as a proportion of total fatty acids within the indicated lipid class or classes (e.g., mole percent composition of the fatty acid or fatty acids in the one or more lipid classes). In some embodiments, for markers in the AC lipid class, the indicated fatty acid-carnitine esters are quantified in absolute terms (e.g. nMoles per gram of plasma or serum). In some embodiments, the level of the first metabolite marker is indicative of the presence, absence, or severity of the diabetic condition. In some embodiments, the first (and/or second, third, fourth, fifth, sixth, seventh, eighth, ninth, and/or tenth) metabolite marker is selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, and CE18:2n6. In some embodiments, the first (and/or second, third, fourth, fifth, sixth, seventh, eighth, ninth, and/or tenth) metabolite marker is selected from the group consisting of AC6:0, AC16:0, AC14:0, AC8:0, AC10:0, AC12:0, TG14:0, FA16:1n7, FA18:1n9, CE16:1n7, PC16:1n7 and PC18:1n9. In some embodiments, the first (and/or second, third, fourth, fifth and/or sixth) metabolite marker is selected from the group consisting of AC6:0, AC8:0, AC10:0, TG14:0, FA16:1n7, and/or PC18:1n9. In some embodiments, the diabetic condition is impaired glucose tolerance, insulin resistance, hepatic steatosis, non-alcoholic steatohepatitis (NASH), pediatric NASH, obesity, childhood obesity, metabolic syndrome, polycystic ovary disease, or gestational diabetes. In some embodiments, the diabetic condition is diabetes. In some embodiments, the diabetic condition is a prediabetic condition (e.g., prediabetes). In some embodiments, the diabetic condition is impaired glucose tolerance or is insulin resistance. In some embodiments, the sample is blood, plasma, serum, or isolated lipoprotein fraction. In some embodiments, measuring the level of the first metabolite marker includes chromatography, immunoassay, enzymatic assay, or mass spectroscopy. In some embodiments, the method of assessing the diabetic condition is a method of diagnosing, identifying, monitoring, and/or assessing the severity of the diabetic condition, and/or assessing the progression or regression of the diabetic condition. In some embodiments, the methods further comprise (1) determining one or more risk factors for the diabetic condition, and correlating the risk factor with the presence, risk of developing, or severity of the diabetic condition; or (2) measuring the level of an additional biomarker, and correlating the level of the additional biomarker with the presence, risk of developing, or severity of the diabetic condition.

In another aspect, the invention provides a method of assessing the response of a subject having a diabetic condition to a treatment for the diabetic condition, comprising measuring the level of a metabolite marker in a sample from the subject following administration of the treatment to the subject, wherein the one or more metabolite markers are selected from the group consisting of: the relative amount of 14:0 to total fatty acid content in TG; the relative amount of 14:0 to total fatty acid content in total lipids; the relative amount of 16:0 to total fatty acid content in PC; the relative amount of 16:0 to total fatty acid content in TG; the relative amount of 16:0 to total fatty acid content in total lipids; the relative amount of 16:1n7 to total fatty acid content in PC; the relative amount of 16:1n7 to total fatty acid content in CE; the relative amount of 16:1n7 to total fatty acid content in TG; the relative amount of 16:1n7 to total fatty acid content in FA; the relative amount of 16:1n7 to total fatty acid content in total lipids; the relative amount of 18:1n9 to total fatty acid content in PC; the relative amount of 18:1n9 to total fatty acid content in CE; the relative amount of 18:1n9 to total fatty acid content in total lipids; the relative amount of 20:3n9 to total fatty acid content in PC; the relative amount of 20:3n9 to total fatty acid content in CE; the relative amount of 20:3n9 to total fatty acid content in TG; the relative amount of 20:3n9 to total fatty acid content in total lipids; the relative amount of 20:3n6 to total fatty acid content in PC; the relative amount of 20:3n6 to total fatty acid content in CE; the relative amount of 20:3n6 to total fatty acid content in total lipids; the relative amount of 18:1n9 to total fatty acid content in FA; the relative amount of 22:6n3 to total fatty acid content in PC; the relative amount of 22:6n3 to total fatty acid content in CE; the relative amount of 22:6n3 to total fatty acid content in TG; the relative amount of 22:6n3 to total fatty acid content in total lipids; the relative amount of 18:0 to total fatty acid content in PC; the relative amount of 18:0 in total lipids to total fatty acid content; the relative amount of 18:2n6 to total fatty acid content in PC; the relative amount of 18:2n6 to total fatty acid content in CE; the relative amount of 18:2n6 to total fatty acid content in FA; the relative amount of 18:2n6 to total fatty acid content in TG; the relative amount of 18:2n6 to total fatty acid content in total lipids; the relative amount of 18:3n6 to total fatty acid content in PC; the relative amount of 18:3n6 to total fatty acid content in CE; the relative amount of 18:3n6 to total fatty acid content in TG; the relative amount of 18:3n6 to total fatty acid content in total lipids; the relative amount of 20:3n6 to total fatty acid content in PC; the relative amount of 20:3n6 to total fatty acid content in CE; and the relative amount of 20:3n6 to total fatty acid content in total lipids. In some embodiments, the method further comprises measuring the level of a second, third, fourth, fifth, sixth, seventh, eighth, ninth, and/or tenth marker from the group. Typically, the relative amount of the fatty acid to total fatty acid content in the lipid of the one or more lipid classes in the sample is calculated as the mole percent composition of the fatty acid in the one or more lipid classes. In some embodiments, the level(s) of the marker(s) are indicative of the presence or absence of the diabetic condition. In some embodiments the level(s) of the marker(s) are indicative of the severity of the diabetic condition. In some embodiments, the diabetic condition is impaired glucose tolerance, insulin resistance, insulin sensitivity, hepatic steatosis, non-alcoholic steatohepatitis (NASH), pediatric NASH, obesity, childhood obesity, metabolic syndrome, polycystic ovary disease, or gestational diabetes. In some embodiments, the diabetic condition is impaired glucose tolerance or insulin resistance. In some embodiments, the sample is blood, plasma, serum, or isolated lipoprotein fraction. In some embodiments, the measurement of the marker or markers comprises chromatography, an immunoassay, an enzymatic assay, or mass spectroscopy. In some embodiments, the treatment of the diabetic condition comprises administration of a PPARs-gamma agonist, a PPARs-alpha agonist, and/or a PPARs-delta agonist.

In still another aspect, the invention provides a method for identifying or monitoring a diabetic condition comprising measuring the level of a first metabolite marker in a sample of a subject wherein the first metabolite marker is selected from the group consisting of 15:0, 16:0, 16:1n7, 18:0, 18:1n7, 18:1n9, 18:2n6, 18:3n6, 20:0, 20:2n6, 20:3n6, 20:3n9, 20:4n3, 20:4n6, 22:2n6, 22:4n6, 22:5n3, 24:0, 24:1n9, FAn3, CEn6, Pen6, PCn7, CEn7, TGn7, PCn9, CEn9, FAn9, PUFA, MUFA, SAT, PCLC, TGLC, PELC, LYLC, and DGLC and wherein the level of the first metabolite marker is characteristic of a diabetic condition. In some embodiments, the level of the first metabolite marker is the level of the first metabolite marker in a class of metabolites. In some embodiments, the first metabolite marker is 16:1n7, 18:1n9, dm18:1n7, t18:2n6, 20:0, 20:3n9, 20:4n3, 20:4n6, 22:5n3, PUFA, or MUFA and the level of the first metabolite marker is the level of the first metabolite marker in phosphatidylcholine. In some embodiments, the first metabolite marker is 22:2n6 or 22:4n6 and the level of the first metabolite marker is the level of the first metabolite marker in triacylglycerol. In some embodiments, the first metabolite marker is 16:0, 16:1n7, 18:1n9, 20:2n6, 20:3n9, 20:4n6, 22:2n6, PUFA or MUFA and the level of the first metabolite marker is the level of the first metabolite marker in cholesterol ester. In some embodiments, the first metabolite marker is 18:1n7, 20:3n6, 22:4n6, 22:5n3, or 24:1n9 and the level of the first metabolite marker is the level of the first metabolite marker in LY. In some embodiments, the first metabolite marker is 16:0 or 20:0 and the level of the first metabolite marker is the level of the first metabolite marker in sphingomyelin. In some embodiments, the first metabolite marker is 15:0, 18:0, or SAT and the level of the first metabolite marker is the level of the first metabolite marker in 1,2-diacylglyceride. In some embodiments, the first metabolite marker is 18:1n9 or 24:0 and the level of the first metabolite marker is the level of the first metabolite marker in free fatty acid. In some embodiments, the first metabolite marker is 18:3n6, 20:3n6, or 20:3n9 and the level of the first metabolite marker is the level of the first metabolite marker in phosphatidylethanolamine. In some embodiments, the subject is administered a therapeutic agent and wherein the level of the first metabolite marker is indicative of the efficacy of the therapeutic agent for the treatment of the diabetic condition. In some embodiments, the subject is under a regimen and wherein the level of the first metabolite marker is indicative of the effect of the regimen for the treatment of the diabetic condition. In some embodiments, the method further comprises measuring the level of a second, third, and/or fourth metabolite marker selected from the group consisting of PC16:1n7, PC18:1n9, PCt18:2n6, PCdm18:1n7, PC20:0, PC20:4n6, PC20:3n9, PC20:4n3, PC22:5n3, PCn9, PCn7, PCMUFA, PCLC, CEn7, CEn9, CE16:1n7, CE18:1n9, CE20:3n9, CE20:2n6, CEMUFA CEn6, CE16:0, CE20:4n6, CE22:2n6, CEPUFA, SP20:0, TG22:2n6, TG22:4n6, PCn6, PCPUFA, FAn9, FA18:1n9, LY22:4n6, LY22:5n3, LY18:1n7, LY20:3n6, LY24:1n9, LYLC, DGSAT, DG15:0, DG18:0, DGLC, PE18:3n6, PE20:3n6, PE20:3n9, PELC, TGn7, TGLC, FAn3, FA24:0, and SP16:0, wherein the level of the first, second, third, and/or fourth metabolite marker are characteristic of a diabetic condition. In some embodiments, the diabetic condition is steatosis, insulin resistance, or type 2 diabetes, prediabetes, insulin resistance, insulin sensitivity, metabolic syndrome, hyperinsulinemia, hepatic steatosis, muscle steatosis, hyperlipidemia, hypercholesterolemia. In some embodiments, the sample is blood, tissue, or plasma, serum, urine, or cerebral spinal fluid. Relevant tissues include adipose, muscle, kidney, liver, vascular endothelium. In some embodiments, measuring the level of the first metabolite marker includes chromatography, immunoassay, enzymatic assay, and mass spectroscopy.

In some embodiments of each of the aforementioned aspects, as well as other aspects described herein, the subject is a mammal such as a human or domesticated animal. In one embodiment, the mammal is a primate. In one embodiment, the mammal is a human. In one embodiment, the subject is being evaluated for bariatric surgery or has had bariatric surgery. In one embodiment, the subject is being monitored for weight loss.

In another aspect of the invention, kits for use in the methods of the invention are provided. In one embodiment, the kit comprises (a) an antibody to the fatty acid; and (b) instructions for use. In one embodiment, the kit further comprises: (c) a second antibody to a second fatty acid. In one embodiment, the kit further comprises: (d) a third antibody to a third fatty acid.

Where aspects or embodiments of the invention are described herein in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

### BRIEF DESCRIPTION OF THE DRAWINGS

FIG. 1. ROC curves for selected metabolites. The metabolite measurements used in these analyses were from fasted plasma and were used to predict glucose intolerance. The AUC measure at the bottom of each graph is the area under the curve and represents the efficacy of predicting glucose intolerance. As is clear from the figures, many lipid metabolites were better predictors of AUC glucose than fasted glucose measures (lower right).

FIG. 2. ROCs for the Prediction of Glucose Intolerance by TG14:0 Alone (Left Panel) and in Combination with Fasted Glucose (Right Panel). The AUC was significantly improved by combining TG14:0 and fasted glucose over both TG14:0 and fasted glucose alone. The gray line in the left panel is the ROC for fasted glucose alone.

### DETAILED DESCRIPTION OF THE INVENTION

In one aspect, the invention provides a method for assessing a diabetic condition in a subject comprising measuring the level of one or more metabolite marker in a sample from a subject. In some embodiments, the one or more metabolite markers are selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, CE18:1n9, PC18:1n9, and PE16:0. In some embodiments, the method comprises a step of correlating the levels of the one or more markers with the presence, absence, risk of developing, progression, regression, and/or severity of the diabetic condition.

In another aspect, the invention provides a method of assessing the response of a subject having a diabetic condition to a treatment for the diabetic condition comprising measuring the level of one or more metabolite markers in a sample from the subject following administration of the treatment to the subject. In some embodiments, the one or more metabolite markers are selected from the group consisting of: the relative amount of 14:0 to total fatty acid content in TG; the relative amount of 14:0 to total fatty acid content in total lipids; the relative amount of 16:0 to total fatty acid content in PC; the relative amount of 16:0 to total fatty acid content in TG; the relative amount of 16:0 to total fatty acid content in total lipids; the relative amount of 16:1n7 to total fatty acid content in PC; the relative amount of 16:1n7 to total fatty acid content in CE; the relative amount of 16:1n7 to total fatty acid content in TG; the relative amount of 16:1n7 to total fatty acid content in FA; the relative amount of 16:1n7 to total fatty acid content in total lipids; the relative amount of 18:1n9 to total fatty acid content in PC; the relative amount of 18:1n9 to total fatty acid content in CE; the relative amount of 18:1n9 to total fatty acid content in total lipids; the relative amount of 20:3n9 to total fatty acid content in PC; the relative amount of 20:3n9 to total fatty acid content in CE; the relative amount of 20:3n9 to total fatty acid content in TG; the relative amount of 20:3n9 to total fatty acid content in total lipids; the relative amount of 20:3n6 to total fatty acid content in PC; the relative amount of 20:3n6 to total fatty acid content in CE; the relative amount of 20:3n6 to total fatty acid content in total lipids; the relative amount of 18:1n9 to total fatty acid content in FA; the relative amount of 22:6n3 to total fatty acid content in PC; the relative amount of 22:6n3 to total fatty acid content in CE; the relative amount of 22:6n3 to total fatty acid content in TG; the relative amount of 22:6n3 to total fatty acid content in total lipids; the relative amount of 18:0 to total fatty acid content in PC; the relative amount of 18:0 in total lipids to total fatty acid content; the relative amount of 18:2n6 to total fatty acid content in PC; the relative amount of 18:2n6 to total fatty acid content in CE; the relative amount of 18:2n6 to total fatty acid content in FA; the relative amount of 18:2n6 to total fatty acid content in TG; the relative amount of 18:2n6 to total fatty acid content in total lipids; the relative amount of 18:3n6 to total fatty acid content in PC; the relative amount of 18:3n6 to total fatty acid content in CE; the relative amount of 18:3n6 to total fatty acid content in TG; the relative amount of 18:3n6 to total fatty acid content in total lipids; the relative amount of 20:3n6 to total fatty acid content in PC; the relative amount of 20:3n6 to total fatty acid content in CE; and the relative amount of 20:3n6 to total fatty acid content in total lipids.

Additional aspects of the invention are provided herein.

### DEFINITIONS

“A”, “an” and “the” include plural references unless the context clearly dictates otherwise.

As used herein “body fluid” includes, but is not limited to, fluids such as blood, plasma, serum, isolated lipoprotein fractions, saliva, urine, lymph, cerebrospinal fluid, and bile.

“Lipid class”, as used herein, indicates classes of lipids such as, for example, neutral lipids, phospholipids, free fatty acids, total fatty acids, triglycerides, cholesterol esters, phosphatidylcholines, phosphatidylethanolamines, diglycerides, lysophatidylcholines, free cholesterol, monoacylglyerides, phosphatidylglycerol, phosphatidylinositol, phosphatidylserine, and sphingomyelin.

Chemical terms, unless otherwise defined, are used as known in the art.

It is understood that wherever embodiments are described herein with the language “comprising,” otherwise analogous embodiments described in terms of “consisting of” and/or “consisting essentially of” are also provided.

As used herein, metabolites (or other biomarkers) that are “positively associated” or “positively correlated” with a condition or disorder include those metabolites whose levels or concentrations generally increase with the disorder relative to normal control subjects or a normal control reference. Metabolites (or other biomarkers) that are “negatively associated” or “negatively correlated” with a condition or disorder generally include those metabolites whose levels or concentrations decrease with the disorder relative to normal control subjects or a normal control reference.

Again, where aspects or embodiments of the invention are described herein in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

### Quantitative Surrogate Markers for Diabetic Conditions

In some embodiments, the invention provides a method of assessing a diabetic condition. In some embodiments, the assessment of the diabetic condition comprises diagnosing, classifying, identifying, monitoring, determining the likelihood of risk of developing, determining the degree (or severity), and/or assessing the progression and/or regression of the diabetic condition. In some embodiments, the diabetic condition is a prediabetic condition. In some embodiments, the diabetic condition is insulin resistance. In some embodiments, the diabetic condition is impaired glucose tolerance. (The term “impaired glucose tolerance” is used interchangeably herein with “glucose intolerance.”) In some embodiments, the diabetic condition is impaired fasting glucose. In some embodiments, the diabetic condition is prediabetes. In some embodiments, the diabetic condition is a form of diabetes.

In some embodiments, the invention provides testing methods that can be used to diagnose, classify, and/or monitor patients with diabetic conditions wherein the condition is selected from the group consisting of: diabetes, type 2 diabetes, insulin resistance, impaired glucose tolerance, impaired fasting glucose, prediabetes, metabolic syndrome, hepatic steatosis, insulin sensitivity, hyperinsulinemia, hepatic steatosis, muscle steatosis, hyperlipidemia, hypercholesterolemia. In some embodiments, the invention provides testing methods that can be used to diagnose, classify, and/or monitor patients with diabetic conditions wherein the condition is selected from the group consisting of: oral glucose intolerance, insulin resistance, insulin sensitivity, hepatic steatosis, type 2 diabetes, and gestational diabetes. In some embodiments, the invention provides testing methods that can be used to diagnose, classify, and/or monitor patients with diabetic conditions wherein the condition is oral glucose intolerance or insulin resistance. In some further embodiments, the invention provides testing methods that can be used to diagnose, classify, and/or monitor patients with diabetic conditions wherein the condition is selected from the group consisting of: non-alcoholic steatohepatitis (NASH), pediatric NASH, obesity, childhood obesity, metabolic syndrome, and polycystic ovary disease.

Diabetes and its related comorbidities and conditions are largely due to changes in the metabolism of lipids. The inventors have discovered that particular amounts of specific lipid metabolites in body fluids correlate with the diabetic condition.

In some aspects, the invention provides metabolic markers for oral glucose intolerance. Impaired glucose tolerance and impaired fasting glucose are known to be pre-diabetic states (Lin et al., Tohoku J. Exp. Med., 212:349-57 (2007)). Impaired oral glucose tolerance has been reported as being a predictor of non-alcoholic fatty liver disease in obese children (Sartorio et al., Eur. J. Clin. Nutr., 61:877-83 (2007)) and of steatoheptatitis and fibrosis in patients with non-alcoholic fatty liver disease (Haukeland et al., Scand. J. Gastroenterol. 40:1469-77 (2005)). The use of oral glucose tolerance testing (OGTT) for detection of gestational diabetes has also been reported (Lapolla et al., J. Clin. Endocrinol. Metab., 2007 Dec. 18 [Epub ahead of print]). In addition, oral glucose intolerance and insulin sensitivity have been linked to Polycystic Ovary Syndrome (Amato et al., Clin Endocrinol. (Oxf), 2007 Nov. 22 [Epub ahead of print]).

In some embodiments, the markers of the invention are used as a substitute for an existing test used to assess a diabetic condition (e.g. fasting blood glucose level or oral glucose tolerance test (OGTT)). In other embodiments, the markers of the invention are used in a test to identify or select a subject for further testing for the diabetic condition via another method including, but not limited to fasting blood glucose level or OGTT.

### Mole Percentage Fatty Acid Compositions as Surrogates for Diabetic Conditions

A lipid metabolite that is a relative proportion of a triglyceride (or any other lipid class) can be measured in a body fluid, such as serum or plasma, as a quantitative measure of the relative proportion of that lipid metabolite in hepatic triglycerides (or other lipid class). If this relative proportion of lipid metabolite (or a collection of lipid metabolites) correlates with insulin resistance, it serves as a quantitative surrogate of the insulin resistance. Thus, the mole percentage of a particular fatty acid within a particular lipid class may be used as a quantitative surrogate for insulin resistance.

In one embodiment, the mole percentage of a single lipid metabolite may be used in the methods of the invention. In other embodiments, mole percentages of two or more lipid metabolites may be used in the methods of the invention, for example, 2, 3, 4, 5, 10, 15, 20, or more lipid metabolites.

According to the present invention, when analyzing the effects rendered by two or more lipid metabolites, one can either evaluate the effects of these lipid metabolites individually or obtain the net effect of these lipid metabolites, e.g., by using various mathematical formulas or models to quantify the effect of each lipid metabolite. A formula containing the levels of one or more lipid metabolites as variables includes any mathematical formula, model, equation, or expression established based on mathematic or statistical principles or methods using the values of one or more lipid metabolites as variables.

In general, any suitable mathematic analyses can be used to analyze the net effect of two or more lipid metabolites with respect to projecting the diabetic condition of a subject. For example, methods such as multivariate analysis of variance, multivariate regression, multiple regression can be used to determine relationships between dependent variables, and independent variables. Clustering, including both hierarchical and nonhierarchical methods, as well as nonmetric Dimensional Scaling can be used to determine associations among variables and among changes in those variables.

In addition, principle component analysis is a common way of reducing the dimension of studies, and can be used to interpret the variance-covariance structure of a data set. Principle components may be used in such applications as multiple regression and cluster analysis. Factor analysis is used to describe the covariance by constructing “hidden” variables from the observed variables. Factor analysis may be considered an extension of principle component analysis, where principle component analysis is used as parameter estimation along with the maximum likelihood method. Furthermore, simple hypothesis such as equality of two vectors of means can be tested using Hotelling's T squared statistic.

In one embodiment, a formula containing one or more lipid metabolites as variables is established by using regression analyses, e.g., multiple linear regressions. Examples of formulas developed include, without any limitation, the following:

k+k₁(FA₁)+k₂(FA₂)+k₃(FA₃) Formula I

k−k₁(FA₁)+k₂(FA₂)+k₃(FA₃) Formula II

k+k₁(FA₁)−k₂(FA₂)+k₃(FA₃) Formula III

k+k₁(FA₁)+k₂(FA₂)−k₃(FA₃) Formula IV

k−k₁(FA₁)−k₂(FA₂)+k₃(FA₃) Formula V

k+k₁(FA₁)−k₂(FA₂)−k₃(FA₃) Formula VI

k−k₁(FA₁)+k₂(FA₂)−k₃(FA₃) Formula VII

k−k₁(FA₁)−k₂(FA₂)−k₃(FA₃) Formula VIII

The formulas may use one or more lipid metabolites as variables, such as 1, 2, 3, 4, 5, 10, 15, 20, or more lipid metabolites. The constants of these formulas can be established by using a set of data obtained from known diabetic conditions. Usually the levels of lipid metabolites used in these formulas can be either the levels at a time point or changes of levels over a period of time.

According to the invention, mathematic formulas established using lipid metabolites can be used to either qualitatively or quantitatively assess the diabetic condition of a subject over a period of time. For example, a formula having one or more lipid metabolites as variables can be used to directly calculate the diabetic condition of a subject. In addition, the net value of a formula containing one or more lipid metabolites can be compared to the standard value of such formula corresponding to a diabetic condition pattern, e.g. progression or regression of a diabetic condition, and the results of such comparison can be used to project diabetic condition development. Specifically, a subject having a net value of a formula similar to or within the range of the standard value of such formula that is assigned to or associated with a progression of a diabetic condition is likely to experience a progression over a period of time. Similarly, a subject having a net value of a formula similar to or within the range of the standard values of such formula that is assigned to or associated with a regression is likely to experience a regression of their diabetic condition over a period of time.

### Additional Quantitative Surrogates for Diabetic Condition

Models of lipid metabolites other than mole percentage may be used as surrogate markers for a diabetic condition. For example see the list of additional biomarkers, e.g. eicosanoids.

### Lipid Metabolites and Additional Biomarkers for Diabetic Conditions

In some embodiments, one or more lipid metabolites are used as metabolite markers for assessing diabetic conditions. In some other embodiments, the metabolite markers used comprise both lipid metabolites and additional biomarkers.

In one embodiment, lipid metabolites include a fatty acid present within a particular lipid class. In one embodiment, the lipid class is selected from the group consisting of neutral lipids, phospholipids, free fatty acids, total fatty acids, triglycerides, cholesterol esters, phosphatidylcholines, and phosphatidylethanolamines. In one embodiment, the lipid class is free fatty acids. In one embodiment, the lipid class is total fatty acids. In one embodiment, the lipid class is triglycerides. In one embodiment, the lipid class is cholesterol esters. In one embodiment, the lipid class is phosphatidylcholines. In one embodiment, the lipid class is phosphatidylethanolamines. In one embodiment, the lipid metabolite is selected from the fatty acids shown in Table 1. The method may involve measuring the amount of more than one lipid metabolite, such as 2, 3, 4, 5, 10, 15, 20, or more lipid metabolites. In one embodiment, two or more lipid metabolites in Table 1 are measured. In one embodiment, three or more lipid metabolites in Table 1 are measured.

In one embodiment, the lipid metabolite is positively correlated with a diabetic condition. In one embodiment, the lipid metabolite is negatively correlated with diabetic condition. In one embodiment, the lipid metabolite is measured as a relative amount within that particular lipid class. In one embodiment, the lipid metabolite is measured in a blood-based body fluid, such as blood, plasma, serum, or lipoprotein fractions.

TABLE 1
Blood-based Lipid Metabolite Markers of Diabetic Condition

| | | | | |
|---|---|---|---|---|
| CE14.0 | FA16.0 | PC14.0 | PE16.0 | TG20.2n6 |
| CE16.0 | FA16.1n7 | PC16.1n7 | PE20.0 | TG20.3n6 |
| CE20.0 | FA18.0 | PC18.0 | PE16.1n7 | TG20.3n9 |
| CE16:1n7 | FA18.1n9 | PC15.0 | PE18.1n9 | TG22.2n6 |
| CE18.1n7 | FA18.1n7 | PC18.1n7 | PE18:3n6 | TG22:4n6 |
| CE18.1n9 | FA18.2n6 | PC18.1n9 | PE20.0 | CETotal.LC |
| CE18.2n6 | FA20.4n6 | PC18.2n6 | PE20.1n9 | TGTotal.LC |
| CE18.3n6 | FA22.2n6 | PC18.3n6 | PE20:3n9 | DGTotal.LC |
| CE22:2n6 | FA22.4n6 | PC18.3n3 | PE20:3n6 | FSTotal.LC |
| CE20.3n9 | FA20.5n3 | PC20.1n9 | PE20.4n6 | AC6:0 |
| CE22.5n6 | FA22.6n3 | PC20:3n9 | PE20.5n3 | AC16:0 |
| DG16:0 | FA24.1n9 | PC20:4n3 | PEdm16.0 | AC14:0 |
| DG18.0 | LY18.0 | PC20.2n6 | PEdm18.0 | AC8:0 |
| DG18.2n6 | LY16.1n7 | PC20.4n6 | TG14.0 | AC10:0 |
| DG18.3n6 | LY18:1n7 | PC22.4n6 | TG14.1n5 | AC3:0 |
| DG20:0 | LY18.1n9 | PC22.5n3 | TG16.0 | AC12:0 |
| DG20.3n6 | LY20.3n9 | PCdm16.0 | TG20.0 | L-Carnitine |
| DG20.3n9 | LY18.2n6 | PCdm18.0 | TG16.1n7 | AC4:0 |
| DG22.1n9 | LY20:3n6 | PCdm18.1n9 | TG18.1n7 | |
| FA14.0 | LY22:4n6 | PCdm18:1n7 | TG18.1n9 | |
| FA15.0 | LY22:5n3 | PE14.0 | TG18.2n6 | |

In some embodiments, the marker(s) for the diabetic condition(s) comprise one or more, two or more, three or more, four or more, five or more, or six or more markers selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, CE18:1n9, PC18:1n9, and PE16:0. In some embodiments, the marker(s) for the diabetic condition(s) comprise one or more, two or more, three or more, four or more, five or more, or six or more markers selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, and PE16:0. In some embodiments, the marker(s) for the diabetic condition(s) comprise one or more, two or more, three or more, four or more, five or more, or six or more markers selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, and CE18:2n6.

In some embodiments, the marker(s) for the diabetic conditions do not comprise a fatty acid within the lipid class of cholesterol esters. In some embodiments, the marker(s) for the diabetic conditions do not comprise a fatty acid within total phospholipids. In some embodiments, the marker(s) for the diabetic condition(s) do not include one or more of the following markers: CE14:0, CE16:1n-7; and CE18:2n-6.

In some embodiments, marker(s) positively associated with the diabetic condition(s) include one or more, two or more, three or more, four or more, five or more, or six or more markers selected from the group consisting of AC6:0, PE20:4n6, AC16:0, AC14:0, FA22:2n6, AC8:0, AC10:0, AC3:0, CETotal:LC, AC12:0, TGTotal:LC, PC18:0, L-Carnitine, PE20:0, DGTotal:LC, AC4:0, TG18:1n9, PE20:4n6, PC20:4n6, CE14:0, CETotal:LC, FSTotal:LC, and PCdm. In some embodiments, the marker(s) positively associated with the diabetic condition(s) that are measured are all medium to long-chain acylcarnitines. In some embodiments, the marker(s) positively associated with the diabetic condition(s) that are measured include one or more, two or more, three or more, four or more, five or more, or six or more markers selected from the group consisting of AC6:0, AC16:0, AC14:0, AC8:0, AC10:0 and AC12:0. A higher value of AC6:0, AC16:0, AC14:0, AC8:0, AC10:0 and/or AC12:0 is associated with a more pronounced diabetic state or increased risk. In some embodiments, the marker(s) positively associated with the diabetic condition(s) that are measured comprise AC6:0, AC8:0, and/or AC10:0.

In some embodiments, marker(s) negatively associated with the diabetic condition(s) include one or more, two or more, three or more, four or more, five or more, or six or more markers selected from the group consisting of TG14:0, PE16:1n7, PE20:0, PC18:2n6, DG18:0 and CE18:2n6, CE16:1n7, CE18:1n9, PC16:1n7, PC18:1n9, FA16:1n7, FA18:1n9, TG16:1n7, and PE16:0. In some embodiments, the marker(s) negatively associated with the diabetic condition(s) that are measured include one or more, two or more, three or more, four or more, five or more, or six or more markers selected from the group consisting of TG14:0, FA16:1n7, FA18:1n9, CE16:1n7, PC16:1n7 and PC18:1n9. A lower value of TG14:0, FA16:1n7, FA18:1n9, CE16:1n7, PC16:1n7 and/or PC18:1n9 is associated with a more pronounced diabetic state or increased risk. In some embodiments, the marker(s) negatively associated with the diabetic condition(s) that are measured comprise TG14:0, FA16:1n7, and/or PC18:1n9.

In some embodiments, the marker(s) for glucose intolerance (i.e., impaired glucose tolerance) comprise one or more, two or more, three or more, four or more, five or more, or six or more markers selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, and PE16:0. In some embodiments, the marker(s) for glucose intolerance comprise one or more, two or more, three or more, four or more, five or more, or six or more markers selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, and CE18:2n6.

In some embodiments, the marker(s) positively associated with oral glucose intolerance and/or glucose AUC include one or more, two or more, three or more, four or more, five or more, or six or more markers selected from the group consisting of AC6:0, PE20:4n6, AC16:0, AC14:0, FA22:2n6, AC8:0, AC10:0, AC3:0, CETotal:LC, AC12:0, TGTotal:LC, PC18:0, L-Carnitine, PE20:0, DGTotal:LC, AC4:0, TG18:1n9, PE20:4n6, PC20:4n6, CE14:0, CETotal:LC, FSTotal:LC, and PCdm. In some embodiments, the marker(s) positively associated with oral glucose intolerance and/or glucose AUC that are measured are all medium to long-chain acylcarnitines. In some embodiments, the marker(s) positively associated with oral glucose intolerance and/or glucose AUC that are measured include one or more, two or more, three or more, four or more, five or more, or six or more markers selected from the group consisting of AC6:0, AC16:0, AC14:0, AC8:0, AC10:0 and AC12:0. A higher value of AC6:0, AC16:0, AC14:0, AC8:0, AC10:0 and/or AC12:0 is associated with a more pronounced diabetic state or increased risk. In some embodiments, the marker(s) positively associated with oral glucose intolerance and/or glucose AUC that are measured comprise AC6:0, AC8:0, and/or AC10:0.

In some embodiments, the marker(s) negatively associated with oral glucose intolerance and/or glucose AUC include one or more, two or more, three or more, four or more, five or more, or six or more markers selected from the group consisting of TG14:0, PE16:1n7, PE20:0, PC18:2n6, DG18:0 and CE18:2n6, CE16:1n7, CE18:1n9, PC16:1n7, PC18:1n9, FA16:1n7, FA18:1n9, TG16:1n7, and PE16:0. In some embodiments, the marker(s) negatively associated with oral glucose intolerance and/or glucose AUC that are measured include one or more, two or more, three or more, four or more, five or more, or six or more markers selected from the group consisting of TG14:0, FA16:1n7, FA18:1n9, CE16:1n7, PC16:1n7 and PC18:1n9. A lower value of TG14:0, FA16:1n7, FA18:1n9, CE16:1n7, PC16:1n7 and/or PC18:1n9 is associated with a more pronounced diabetic state or increased risk. In some embodiments, the marker(s) negatively associated with oral glucose intolerance and/or glucose AUC that are measured comprise TG14:0, FA16:1n7, and/or PC18:1n9.

Markers positively correlating with therapeutic improvements in oral glucose intolerance, insulin resistance and/or other diabetic conditions comprise one or more, two or more, three or more, four or more, five or more, or six or more markers selected from the group consisting of the following: the relative amount of 14:0 to total fatty acid content in TG; the relative amount of 14:0 to total fatty acid content in total lipids; the relative amount of 16:0 to total fatty acid content in PC; the relative amount of 16:0 to total fatty acid content in TG; the relative amount of 16:0 to total fatty acid content in total lipids; the relative amount of 16:1n7 to total fatty acid content in PC; the relative amount of 16:1n7 to total fatty acid content in CE; the relative amount of 16:1n7 to total fatty acid content in TG; the relative amount of 16:1n7 to total fatty acid content in FA; the relative amount of 16:1n7 to total fatty acid content in total lipids; the relative amount of 18:1n9 to total fatty acid content in PC; the relative amount of 18:1n9 to total fatty acid content to total fatty acid content in CE; the relative amount of 18:1n9 to total fatty acid content in total lipids; the relative amount of 20:3n9 to total fatty acid content in PC; the relative amount of 20:3n9 to total fatty acid content in CE; the relative amount of 20:3n9 to total fatty acid content in TG; the relative amount of 20:3n9 to total fatty acid content in total lipids; the relative amount of 20:3n6 to total fatty acid content in PC; the relative amount of 20:3n6 to total fatty acid content in CE; and the relative amount of 20:3n6 to total fatty acid content in total lipids. In some embodiments, the relative amount of each of the markers is typically calculated as the mole percentage of the fatty acid (within the indicated lipid class or within total lipids).

In some embodiments, markers negatively correlating with therapeutic improvements in oral glucose intolerance, insulin resistance and/or other diabetic conditions comprise one or more, two or more, three or more, four or more, five or more, or six or more markers selected from the group consisting of the following markers: the relative amount of 18:1n9 to total fatty acid content in FA; the relative amount of 22:6n3 to total fatty acid content in PC; the relative amount of 22:6n3 to total fatty acid content in CE; the relative amount of 22:6n3 to total fatty acid content in TG; the relative amount of 22:6n3 to total fatty acid content in total lipids; the relative amount of 18:0 to total fatty acid content in PC; the relative amount of 18:0 to total fatty acid content in total lipids; the relative amount of 18:2n6 to total fatty acid content in PC; the relative amount of 18:2n6 to total fatty acid content in CE; the relative amount of 18:2n6 to total fatty acid content in FA; the relative amount of 18:2n6 to total fatty acid content in TG; the relative amount of 18:2n6 to total fatty acid content in total lipids; the relative amount of 18:3n6 to total fatty acid content in PC; the relative amount of 18:3n6 to total fatty acid content in CE; the relative amount of 18:3n6 to total fatty acid content in TG; the relative amount of 18:3n6 to total fatty acid content in total lipids; the relative amount of 20:3n6 to total fatty acid content in PC; the relative amount of 20:3n6 to total fatty acid content in CE; and the relative amount of 20:3n6 to total fatty acid content in total lipids. In some embodiments, the relative amount of each of the markers is typically calculated as the mole percentage of the fatty acid (within the indicated lipid class or within total lipids).

The following additional biomarkers may aid the diagnosis of diabetic conditions: (1) malonyl-CoA and malonylcarnitine; (2) free carnitine, and acylcarnitines listed in Table 2; and (3) sterols and bile acids listed in Table 3. Body fluid and cellular samples may be used to measure these additional biomarkers. Examples of cellular samples include, but are not limited to, lymphocytes and macrophages.

TABLE 2
List of Acylcarnitine Metabolites

| | | |
|---|---|---|
| L-Carnitine | Butyrobetaine | Acetyl carnitine |
| Propionyl carnitine | Butyryl carnitine | Hexanoyl carnitine |
| Valeryl carnitine | Octanoyl carnitine | Decanoyl carnitine |
| Myristoyl carnitine | Palmitoyl carnitine | Stearoyl carnitine |
| Oleoyl carnitine | Linoleoyl carnitine | Arachidoyl carnitine |
| Dodecanoyl carnitine | | |

TABLE 3
List of Bile Acid and Sterol Metabolites

| | | |
|---|---|---|
| Cholic Acid | Chenodeoxycholic Acid | Deoxycholic Acid |
| Lithocholic Acid | Glycocholic Acid | Taurodeoxycholate |
| Glycochenodeoxy- | Taurochenodeoxycholate | β-Muricholic Acid |
| cholate | Ursodeoxycholic acid | Taurodeoxycholic acid |
| Taurolithocholic | Glycodesoxycholic acid | Glycolithocholic acid |
| acid | Cholesterol | Coprostanol |
| Taurocholic acid | Lanosterol | Lathosterol |
| Glycoursodeoxycholic | Desmosterol | Campesterol |
| acid | Lathosterol | Campesterol |
| Cholestanol | 4-Cholesten-3-One | Fucosterol |
| Beta-Sitosterol | | |
| Coprosterol | | |
| Stigmasterol | | |

Additionally, the following additional biomarkers may aid in the diagnosis of a diabetic condition: (1) The sterols and bile acids listed in Table 3 (levels increase with increased cholesterol synthesis); (2) Eicosanoids including, but not limited to, those shown in Table 4; and/or (3) Cytokines and chemokines including, but not limited to, TNFalpha, IL-6, leptin, adiponectin. Body fluid and cellular samples may be used to measure the additional markers. Examples of cellular samples include, but are not limited to, lymphocytes and macrophages.

TABLE 4
List of Eicosanoid Metabolites

| | | |
|---|---|---|
| 13-14-dihydro-15-keto PGA2 | PGB2 | PGD2 |
| PGE2 | 6-keto PGF1a | PGF2a |
| 11b-PGF2a | 15-keto PGF2a | PGJ2 |
| 15-deoxy-o-12,14-PGJ2 | TXB2 | 11-dehydro TXB2 |
| 8-iso-PGF2a | 9-HODE | 13-HODE |
| 5-HETE | 8-HETE | 9-HETE |
| 11-HETE | 12-HETE | 15-HETE |
| 5(S)-HEPE | 12(S)-HEPE | 15(S)-HEPE |
| LTB4 | LTB5 | LTC4 |
| LTD4 | LTE4 | LTF4 |
| Lipoxin A4 | 20-HETE | 12(13)-DiHOME |
| 12(13)-EpOME | 9(10)-EpOME | 5(6)-EpETrE |
| 11(12)-EpETrE | 14(15)-EpETrE | 5,6-DiHETrE |
| 8,9-DiHETrE | 11,12-DiHETrE | 14,15-DiHETrE |
| 14,15-DiHETE | 17,18-DiHETE | 14(15)-EpETE |
| 17(18)-EpETE | 19(20)-DiHDPA | |

Measurements of the amounts of one or more of these additional biomarkers may be used in the methods of the invention, in addition to measurement of a lipid metabolite. In one embodiment, the amount of one of the biomarkers is measured in a sample from the subject. In one embodiment, the amounts of two of the biomarkers are measured in a sample from the subject. In other embodiments, 3, 4, 5, 6, 7, 8, 10, 12, 15, 20, or more of the biomarkers may be measured in a sample from the subject.

Diagnostic Cutoff Values for Selected Markers Associated with Oral Glucose Intolerance:

The concentration of AC6:0 expected to provide utility for diagnosing pre-diabetes and other diabetes-related conditions is between 0.44 and 0.70 nMoles per gram of plasma or serum. A higher value is associated with a more pronounced diabetic state or increased risk.

The concentration of AC8:0 expected to provide utility for diagnosing pre-diabetes and other diabetes-related conditions is between 0.119 and 0.260 nMoles per gram of plasma or serum. A higher value is associated with a more pronounced diabetic state or increased risk.

The concentration of AC10:0 expected to provide utility for diagnosing pre-diabetes and other diabetes-related conditions is between 0.123 and 0.315 nMoles per gram of plasma or serum. A higher value is associated with a more pronounced diabetic state or increased risk.

The concentration of PE20:4n6 expected to provide utility for diagnosing pre-diabetes and other diabetes-related conditions is between 21.30 and 24.15 mole percent of total phosphatidylethanolamine fatty acid composition in plasma or serum. A higher value is associated with a more pronounced diabetic state or increased risk.

The concentration of PC18:0 expected to provide utility for diagnosing pre-diabetes and other diabetes-related conditions is between 12.40 and 14.20 mole percent of total phosphatidylcholine fatty acid composition in plasma or serum. A higher value is associated with a more pronounced diabetic state or increased risk.

The concentration of TG14:0 expected to provide utility for diagnosing pre-diabetes and other diabetes-related conditions is between 0.07 and 0.04 mole percent of total triglyceride fatty acid composition in plasma or serum. A higher value is associated with a more pronounced diabetic state or increased risk.

### Methods of Diagnosing and Monitoring

The methods of the invention may be used to diagnose a particular condition, for example diabetes, type 2 diabetes, insulin resistance, impaired glucose tolerance, impaired fasting glucose, prediabetes, metabolic syndrome, hepatic steatosis, insulin sensitivity, hyperinsulinemia, hepatic steatosis, muscle steatosis, hyperlipidemia, hypercholesterolemia. The methods may also be used to assess the severity of a diabetic condition, monitor a diabetic condition, assess the progression or regression of a diabetic condition, and/or monitor the response to a therapy.

For example, a method of diagnosis may comprise determining a relative amount of one or more fatty acids to total fatty acid content in the lipids of one or more lipid classes in a sample from a body fluid of the subject, and correlating that amount with the presence of a diabetic condition. In some embodiments, the method may further comprise the step of comparing the relative amount to a reference, wherein if the relative amount is greater than the reference, diabetes, type 2 diabetes, insulin resistance, impaired glucose tolerance, impaired fasting glucose, prediabetes, metabolic syndrome, hepatic steatosis, insulin sensitivity, hyperinsulinemia, hepatic steatosis, muscle steatosis, hyperlipidemia, hypercholesterolemia. is indicated. In some embodiments, the method may further comprise the step of comparing the relative amount to a reference, wherein if the relative amount is less than the reference, diabetes, type 2 diabetes, insulin resistance, impaired glucose tolerance, impaired fasting glucose, prediabetes, metabolic syndrome, hepatic steatosis, insulin sensitivity, hyperinsulinemia, hepatic steatosis, muscle steatosis, hyperlipidemia, hypercholesterolemia. is indicated.

Similarly, the severity of the diabetic condition may be measured, wherein the relative amount indicates the severity of the diabetic condition. Additionally, the relative amount indicates the current state of the condition, and thus a diabetic condition may be monitored and/or the progression or regression of the condition assessed. The relative amount may be measured at two or more time points. In some embodiments, the relative amount may be measured at 2, 3, 4, 5, 6, 7, 8, 10, 12, 15, 20, or more time points. Each time point may be separated by one or more hours, days, weeks, or months. By measuring the relative amount at more than one time point, the clinician may assess a subject's response to treatment.

### Methods of Measurement of Lipid Metabolites and Biomarkers

Assays for lipid metabolite content may be performed on a body fluid or tissue sample. In one embodiment, the assays may be performed on whole blood, plasma, serum, or isolated lipoprotein fractions. Assays for the additional biomarkers may be performed on a body fluid or a cellular sample. These lipid metabolites and other biomarkers may readily be isolated and/or quantified by methods known to those of skill in the art, including, but not limited to, methods utilizing: mass spectrometry (MS), high performance liquid chromatography (HPLC), isocratic HPLC, gradient HPLC, normal phase chromatography, reverse phase HPLC, size exclusion chromatography, ion exchange chromatography, capillary electrophoresis, microfluidics, chromatography, gas chromatography (GC), thin-layer chromatography (TLC), immobilized metal ion affinity chromatography (IMAC), affinity chromatography, immunoassays, and/or colorimetric assays. In one embodiment, the methods of the invention utilize MS to determine lipid metabolite content. In one embodiment, the methods of the invention utilize an immunoassay to determine lipid metabolite content. In one embodiment, the methods of the invention utilize MS to determine the concentration of a biomarker. In one embodiment, the methods of the invention utilize an immunoassay to determine the concentration of a biomarker.

Various analytical methods are well known to those of skill in the art, and are further described in the following documents, which are herein incorporated by reference in their entirety:

Mass Spectrometry: Cyr et al., J Chromatogr B Analyt Technol Biomed Life Sci. 2006 Feb. 17; 832(1):24-9; Vogeser et al., Clin Chem Lab Med. 2003 February; 41(2):117-26.

HPLC: Khalil et al., J Chromatogr B Analyt Technol Biomed Life Sci. 2006 May 23; Fouassier et al., J Thromb Haemost. 2006 May; 4(5):1136-9; Badiou et al., Clin Lab. 2004; 50(3-4):153-8; Brunelli et al., Clin Lab. 2001; 47(7-8):393-7.

Capillary electrophoresis: Zinellu et al., J Sep Sci. 2006 March; 29(5):704-8; Jabeen et al., Electrophoresis. 2006 May 23; Gao et al., Electrophoresis. 2006 May; 27(9):1784-9.

Microfluidics: Johannessen et al., IEEE Trans Nanobioscience. 2002 March; 1(1):29-36; Herrmann et al., Lab Chip. 2006 April; 6(4):555-60; Yang et al., ASAIO J. 2005 September-October; 51(5):585-90; Dupuy et al., Clin Chem Lab Med. 2005; 43(12):1291-302.

Chromatography: Paterson et al., Addiction. 2005 December; 100(12):1832-9; Bottcher et al., J Anal Toxicol. 2005 November-December; 29(8):769-76; Julak, Prague Med Rep. 2005; 106(2):175-94; Boettcher et al., Clin Lab. 2000; 46(1-2):49-52.

Immunoassays: Westermann et al., Clin Lab. 2002; 48(1-2):61-71; Aoyagi et al., Clin Lab. 2001; 47(3-4):119-27; Hubl et al., Clin Lab. 2005; 51(11-12):641-5; Haller et al., J Anal Toxicol. 2006 March; 30(2):106-11; Bayer et al., Clin Lab. 2005; 51(9-10):495-504; Groche et al., Clin Lab. 2003; 49(11-12):657-61; Ivan et al., Clin Lab. 2005; 51(7-8):381-7.

Colormetric assays: Kramer et al., Clin Chem. 2005 November; 51(11):2110-6; Groche et al., Clin Lab. 2003; 49(11-12):657-61; Wolf, Clin Chim Acta. 2006 Mar. 24.

The TrueMass® analytical platform may also be used for the methods of the invention. TrueMass® is an analytical platform that may be used to get quantitative data from serum or plasma on approximately 400 individual metabolites involved in structural and energetic lipid metabolism such as triglyceride, cholesterol ester and phospholipid metabolism. This platform is useful in profiling diseases as structural and energetic lipids are central components of metabolism and integrated into virtually every biological process in the body. A data set for a plasma or serum sample comprises the quantitative measurement of free cholesterol and the following fatty acids from phosphatidylcholines, phosphatidylethanolamines, lysophosphatidylcholines, triglycerides, diglycerides, free fatty acids, and cholesterol esters: 14:0, 15:0, 16:0, 18:0, 20:0, 22:0, 24:0, 14:1n5, 16:1n7, t16:1n7, 18:1n9, t18:1n9, 18:1n7, 18:2n6, t18:2n6, 18:3n6, 18:3n3, 18:4n3, 20:1n9, 20:2n6, 20:3n9, 20:3n6, 20:4n6, 20:3n3, 20:4n3, 20:5n3, 22:1n9, 22:2n6, 22:4n6, 22:5n3, 22:6n3, 24:1n9, 24:6n3 and plasmalogen derivatives of 16:0, 18:0, 18:1n9 and 18:1n7. Methods for using TrueMass® are known to those of skill in the art, and are also described in the following documents, which are herein incorporated by reference in their entirety: U.S. patent application Ser. No. 11/296,829 (filed Dec. 6, 2005; U.S. Patent Publication No. 2006/0084129); Mutch et al., FASEB J. 2005 April; 19(6):599-601; Stone et al., J Biol Chem. 2004 Mar. 19; 279(12):11767-76; Watkins et al., J. Nutr. 2003 November; 133(11):3386-91; Watkins et al., Lipid Res. 2002 November; 43(11):1809-17.

Use of Metabolite Markers in Combination with Other Indicators/Tests

The invention further provides methods of assessing a diabetic condition that optionally comprise evaluating one or more risk indicators, measuring glucose levels, and/or performing another diagnostic test for a diabetic condition, in addition to measuring the level of one or more metabolite markers described herein. A variety of risk indicators for diabetes are known to those skilled in the art and can include, but are not limited to, the following: age, weight, body mass index (BMI), family history (e.g., relatives with diabetes), medical history (e.g., history of gestational diabetes), ethnic background, high blood pressure, cholesterol levels, and activity level. In some embodiments, glucose levels are measured by fasting plasma glucose (FPG). In some alternative embodiments, glucose levels are measured by oral glucose tolerance test (OGTT). In some embodiments, one or more of the metabolite markers used herein are used in combination with a test for glycosylated hemogoblin in the blood (e.g., HbA1c), to assess a diabetic condition.

In some embodiments, the methods, in addition to comprising measuring one or more metabolite markers such as lipid metabolites, further comprise (1) determining the presence or absence of one or more risk factors for the diabetic condition, and correlating the presence or absence of the risk factor with the presence, risk of developing, or severity of the diabetic condition; and/or (2) measuring the level of an additional biomarker, and correlating the level of the additional biomarker with the presence, risk of developing, or severity of the diabetic condition. In some embodiments, the one or more risk factors are selected from the group consisting of: age, weight, body mass index (BMI), family history, medical history, ethnic background, high blood pressure, cholesterol level, and activity level. In some embodiments, the additional biomarker is selected from the group consisting of blood glucose or glycosylated hemoglobin.

### Kits

Kits for practicing the methods of the invention are provided. The kits include (a) one or more reagents for measuring the amount of one or more lipid metabolites (and/or additional biomarkers); and (b) instructions for use. A kit may provide 1, 2, 3, 4, 5, 10, 15, 20, or more reagents for measuring the amount of 1, 2, 3, 4, 5, 10, 15, 20, or more lipid metabolites. The kit may further provide one or more reagents for measuring one or more additional biomarkers, such as those disclosed above, and in Tables 2-4. In one embodiment, the kit includes one or more reagents for use in an immunoassay. In one embodiment, the kit includes one or more reagents for use in an MS assay. The invention is further illustrated by the following nonlimiting examples.

### EXAMPLES

### Example 1

### Lipid Metabolites Provide Improved Assessment of Glucose Intolerance from a Fasted Blood Sample

### Protocol and Methods

Twenty-five volunteers, nine young (five female, four male; ages 20-32 yr) and 16 elderly (11 female, five male; ages 65-74 yr) subjects, were enrolled in the study. Both groups were equally multiracial, with six Caucasians, two Hispanics, and one African-American in the young group and 12 Caucasians, three Hispanics, and one African-American in the elderly group. All volunteers were healthy by history and physical examination, and none were participating in regular aerobic or resistance training routines. Subjects' total cholesterol was less than 250 mg/dl (6.5 mmol/liter), and TSH levels were within the normal range (0.49-4.70 μIU/ml). Further exclusions included palpable liver enlargement; positive hepatitis B, C, or HIV tests; anemia; or elevation in level of more than one of the following: alkaline phosphatase more than 122 U/liter, alanine aminotransferase more than 51 U/liter, or aspartate aminotransferase more than 40 U/liter. Subjects did not use lipid-lowering medications, diabetes medications, anticoagulants, illicit drugs, or consume alcohol in excess (more than one drink per day or six drinks per week).

Volunteers were admitted to the General Clinical Research Center (GCRC) at the University of Texas Medical Branch in the evening, and the next morning, after an overnight fast, MRS was conducted, followed by a full-body dual x-ray absorptiometry (DEXA) scan. Volunteers then returned to the GCRC, where a 20-gauge IV catheter was inserted in the antecubital vein for blood sampling. After two baseline samples, a 2-hour oral glucose tolerance test (OGTT) with 75 mg dextrose was performed. The subjects were fasted for approximately 12 h before the start of the OGTT. Blood was sampled every 30 minutes [52].

### Analytical Methods

The lipids from each sample were extracted in the presence of authentic surrogate standards (described in PCT Application No. PCT/US02/21426, titled “Generating, Viewing, Interpreting, and Utilizing a Quantitative Database of Metabolites”, described at, inter alia, pages 16-17, and 25-28, herein incorporated by reference in its entirety. Briefly, the lipids from plasma (200 ul) were extracted with chloroform:methanol (2:1, v/v) in the presence of authentic internal standards by the method of Folch et al. [53]. Individual lipid classes within the extract were separated by preparative chromatography as described by Watkins et al. [54]. Briefly, for neutral lipids, TLC plates were impregnated with 1 mM EDA, pH 5.5, and washed by ascending development. Sample extracts were dried under nitrogen and spotted onto EDTA-impregnated TLC plates. For the separation of lipid classes [total PL, FFA, TAG, DAG, FC, CE], a solvent system consisting of petroleum ether/diethyl ether/acetic acid (80:20:1, by vol) was employed. The phospholipid classes are separated as described by Lutzke and Braughler (REF1) via high performance liquid chromatography on an Agilent 1100 Series HPLC, with a Phenomenex Sperex 5u OH Diol column (250×4.6 mm, 5 micron) and a SEDEX 75 evaporative light scattering detector. Isolated lipid classes were trans-esterified in 3 N methanolic HCl in a sealed vial under a nitrogen atmosphere at 100° C. for 45 min. The resulting fatty acid methyl esters were extracted with hexane containing 0.05% butylated hydroxytoluene and prepared for gas chromatography by sealing the hexane extracts under nitrogen.s

Fatty acid methyl esters were separated and quantified by capillary gas chromatography using a Hewlett-Packard (Wilmington, Del.) gas chromatograph (model 6890) equipped with a 60 m DB-23 capillary column (J&W Scientific, Folsom, Calif.), a flame-ionization detector, and Hewlett-Packard ChemStation software.

Once a chromatogram was generated, the analytical software (Atlas 2003; Thermo Electron Corporation) identified each analyte lipid metabolite of interest based on the reference standard and generated a raw area. The raw area, peak shape parameters and the response factor for each analyte were exported to an information management system, where an integration algorithm was used to generate the corrected areas for each analyte of interest. Quantitative data were calculated by taking the ratio of the area of the analyte peak to the area of the appropriate surrogate. This ratio was multiplied by the concentration of the surrogate in the original sample to generate data in a microgram per gram of sample format. Each analyte was then divided by its molecular weight and multiplied by 1000 to calculate the nMoles of analyte per gram of sample. Mole percentage data for each lipid class was calculated by dividing the concentration of each fatty acid by the sum of the concentrations of fatty acids within that class.

### Statistical Analysis

The study population included 34 subjects ages 21 to 78 with a wide range of insulin and glucose tolerance. In order to determine the relationship between fatty acids in the blood and glucose intolerance, two outcomes were used. Frank diabetics were excluded from this analysis. The first outcome was the area under the curve (AUC) of the glucose tolerance test (GTT). For determining those lipid metabolites predictive of the AUC of the GTT, each metabolite measurement (at baseline) was examined for its correlation with AUC glucose, incremental AUC glucose, fasted glucose and fasted insulin. The second outcome was the level of glucose in the blood at the two-hour time point. The ability of each analyte to identify glucose intolerant subjects (2-hour glucose>140 mg/dL) was determined by calculating the area under the curve of the ROC. For those metabolites that performed well in predicting glucose resistant subjects, a logistic regression was used to create a measure that combined fasting glucose with the metabolite. The performance of each selected analyte in predicting AUC glucose was evaluated by determining the AUC in a receiver-operator curve using a 2-hour glucose of 140 mg/dL cutoff.

### Rationale for Fasted Lipid Metabolites Predicting Glucose Intolerance

Glucose intolerance (defined as 2-hour glucose post a 75 mg OGTT test of greater than 140 mg/dL) is a better predictor of insulin resistance and insulin resistance-related morbidity and mortality than fasted glucose measures [55]. Although its predictive power is greater, the oral glucose tolerance test is employed infrequently because it is cumbersome, expensive and impractical. Therefore, we attempted to identify plasma lipid metabolites that either correlated with the AUC of the OGTT or predicted glucose intolerance (2-hour glucose>140 mg/dL) from a fasted sample, or that added predictive value to fasted glucose measurements.

Lipomics quantified lipid metabolites from fasted samples prior to a glucose tolerance test in 34 subjects. Lipids measured included acylcarnitines (AC), butyrobetaine, L-carnitine, cholesterol, cholesterol esters (CE), diglycerides (DG), free cholesterol (FS), free fatty acids (FA), lysophosphatidylcholine (LY), phosphatidylcholine (PC), phosphatidylethanolamine (PE) and triglycerides (TG). For CE, DG, FA, LY, PC, PE and TG lipid classes the following fatty acid components were either quantified in absolute terms (nMoles per gram of plasma or serum) or as a proportion of total fatty acids within the lipid class: 14:0, 15:0, 16:0, 18:0, 20:0, 22:0, 24:0, 14:1n5, 16:1n7, 18:1n7, 18:1n9, 20:1n9, 20:3n9, 22:1n9, 24:1n9, 18:2n6, 18:3n6, 20:2n6, 20:3n6, 20:4n6, 22:2n6, 22:4n6, 22:5n6, 18:3n3, 18:4n3, 20:3n3, 20:4n3, 20:5n3, 22:5n3, 22:6n3, 24:6n3, plasmalogen derivatives of 16:0, 18:0, 18:1n7 and 18:1n9, t16:1n7 t18:1n9 t18:2n6. For the AC lipid class, the following fatty acid-carnitine esters were quantified in absolute terms (nMoles per gram of plasma or serum): 2:0, 3:0, 4:0, 5:0, 6:0, 8:0, 10:0, 12:0, 14:0, 16:0, 18:0, 18:1n9, 18:2n6. The term “Total.LC” indicates the value shown is the total concentration of the lipid class expressed as nMoles per gram of serum or plasma. Thus, the abbreviation PC18:2n6 indicates either the absolute amount of 18:2n6 in plasma or serum phosphatidylcholine or the percentage of plasma or serum phosphatidylcholine comprised of linoleic acid (18:2n6), the term AC6:0 indicates the absolute amount of hexanoylcarnitine present in serum or plasma and the term TGTotal.LC indicates the absolute amount of triglyceride present in plasma or serum.

### Results

Fasted levels of many lipid metabolites were independently predictive of AUC glucose at P<0.1 (Table 5). In particular, the acylcarnitines and free carnitine were significantly and positively correlated with AUC glucose. The metabolites AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0 and CE18:2n6 were especially good independent predictors of AUC glucose and may be viewed as potential surrogate markers for diabetic conditions.

TABLE 5
Fasted Lipid Metabolites Significantly Correlated with AUC Glucose at P < 0.10

| | | | Correlation | | Linear Regression: | | | | | |
|---|---|---|---|---|---|---|---|---|---|---|
| | Correlation | | with | | Baseline Glucose + | | Correlation | | Correlation | |
| | with AUC | | Incremental | | Fatty Acid | | with Fasting | | with Fasting | |
| | Glucose (No | | AUC Glucose | | Predicting AUC | | Glucose (No | | Insulin (No | |
| | Diabetics) | | (No Diabetics) | | Glucose | | Diabetics) | | Diabetics) | |
| | Corr. | P | Corr. | P | Baseline | | Corr. | P | Corr. | P |
| Predictors | Coeff. | value | Coeff. | value | Gluc. | Analyte | Coeff. | value | Coeff. | value |
| AC6:0 | 0.57 | 0.00 | 0.07 | 0.70 | — | — | 0.22 | 0.22 | 0.13 | 0.46 |
| PE20.4n6 | 0.52 | 0.00 | 0.24 | 0.17 | 0.00 | 0.00 | 0.09 | 0.62 | −0.08 | 0.64 |
| AC16:0 | 0.50 | 0.00 | −0.04 | 0.81 | — | — | 0.39 | 0.02 | 0.32 | 0.07 |
| AC14:0 | 0.49 | 0.00 | 0.08 | 0.66 | — | — | 0.22 | 0.23 | 0.29 | 0.11 |
| FA22.2n6 | 0.49 | 0.01 | −0.01 | 0.95 | 0.03 | 0.07 | 0.41 | 0.03 | 0.40 | 0.04 |
| AC8:0 | 0.45 | 0.01 | 0.15 | 0.42 | — | — | 0.26 | 0.15 | 0.25 | 0.16 |
| AC10:0 | 0.42 | 0.02 | 0.13 | 0.47 | — | — | 0.22 | 0.22 | 0.26 | 0.15 |
| AC3:0 | 0.39 | 0.02 | −0.01 | 0.96 | — | — | 0.33 | 0.06 | 0.30 | 0.09 |
| CETotal:LC | 0.39 | 0.03 | 0.16 | 0.38 | 0.02 | 0.09 | 0.27 | 0.13 | 0.25 | 0.16 |
| AC12:0 | 0.39 | 0.03 | 0.10 | 0.59 | — | — | 0.27 | 0.14 | 0.25 | 0.17 |
| TG14.0 | −0.38 | 0.03 | −0.12 | 0.51 | 0.00 | 0.01 | 0.10 | 0.59 | 0.25 | 0.16 |
| TGTotal:LC | 0.37 | 0.04 | −0.03 | 0.86 | 0.04 | 0.34 | 0.48 | 0.01 | 0.26 | 0.15 |
| PE16.1n7 | −0.35 | 0.04 | −0.19 | 0.27 | 0.01 | 0.11 | −0.21 | 0.22 | −0.15 | 0.41 |
| PC18.0 | 0.34 | 0.05 | 0.04 | 0.81 | 0.01 | 0.10 | 0.19 | 0.29 | 0.35 | 0.04 |
| L-Carnitine | 0.34 | 0.06 | −0.26 | 0.14 | — | — | 0.47 | 0.01 | 0.14 | 0.44 |
| PE20.0 | −0.33 | 0.06 | −0.14 | 0.43 | 0.00 | 0.02 | 0.03 | 0.85 | −0.09 | 0.60 |
| PC18.2n6 | −0.33 | 0.06 | −0.20 | 0.26 | 0.01 | 0.06 | −0.07 | 0.68 | −0.13 | 0.46 |
| DGTotal:LC | 0.33 | 0.06 | 0.12 | 0.52 | 0.03 | 0.34 | 0.39 | 0.02 | 0.26 | 0.14 |
| AC4:0 | 0.30 | 0.09 | −0.20 | 0.26 | — | — | 0.51 | 0.00 | 0.24 | 0.17 |
| TG18.1n9 | 0.30 | 0.09 | −0.01 | 0.97 | 0.01 | 0.14 | 0.15 | 0.39 | −0.14 | 0.43 |
| DG18.0 | −0.33 | 0.09 | 0.03 | 0.88 | 0.01 | 0.27 | −0.29 | 0.14 | −0.08 | 0.68 |
| CE18.2n6 | −0.29 | 0.09 | 0.03 | 0.86 | 0.01 | 0.25 | −0.24 | 0.17 | −0.04 | 0.84 |

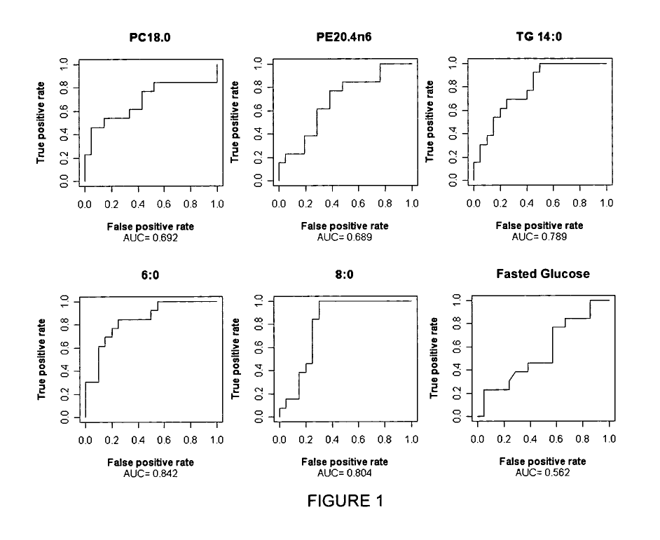

Lipid metabolites showing good correlation between their concentration in fasted samples and AUC glucose were evaluated for their diagnostic potential for glucose intolerance. Using a receiver-operator curve, the ability of the analyte to predict glucose intolerance (2-hour OGTT glucose>140 mg/dL) was determined. Examples of these analyses are shown below. Many lipid metabolites were significantly more predictive of glucose intolerance than fasted glucose measures (FIG. 1), indicating that these metabolites are significant improvements over current practice in diagnosing and predicting diabetic conditions.

As one example, the potential for fasted AC6:0 to serve as a surrogate for AUC glucose is described by the results of an ROC analysis (Table 6a). The quantitative cutoffs for predicting glucose intolerance (as defined by 2-hour glucose over 140 mg/dL) are shown in the left hand column and are expressed in nanomoles of AC6:0 per gram of plasma or serum. AC6:0 and other lipid metabolites were significantly more predictive of glucose intolerance in this population than fasted glucose as assessed by the area under the ROC curve.

TABLE 6a
Details of the ROC Analysis of Fasted AC6:0 in
Predicting AUC Glucose.

| Positive is greater | Sensitivity | | | |
|---|---|---|---|---|
| than or equal to: | (%) | Specificity (%) | NPV (%) | PPV (%) |
| 0.086 | 31 | 100 | 69 | 100 |
| 0.057 | 62 | 90 | 78 | 80 |
| 0.055 | 69 | 85 | 81 | 75 |
| 0.053 | 77 | 80 | 84 | 71 |
| 0.052 | 85 | 75 | 88 | 69 |
| 0.045 | 92 | 50 | 91 | 55 |
| 0.04 | 100 | 45 | 100 | 54 |

A diagnostic test for oral glucose tolerance or other diabetic condition may require more certainty around sensitivity or specificity depending on the application. Thus, diagnostic cutoffs for determining oral glucose intolerance (2h glucose above 140 mg/dL) from a fasted blood sample may differ based on the specific testing requirements. Useful ranges for diagnostic cutoffs for determining oral glucose intolerance are shown in Table 6b. For each selected metabolite the concentration providing the lowest potential useful cutoff is shown under the column titled “Lower Limit” and the highest potential useful cutoff is shown under the column titled “Upper Limit.” A concentration of a marker positively associated with glucose intolerance in a sample from a subject that is higher than the cutoff value indicates the subject is glucose intolerant. The direction of the association indicated in the column on the far right. A positive association indicates that an increase in the concentration of the marker indicates in increased risk or severity of oral glucose intolerance or a diabetic condition. A negative association indicates that an decrease in the concentration of the marker indicates in increased risk or severity of oral glucose intolerance or a diabetic condition.

TABLE 6B
Ranges for useful diagnostic cutoffs for determining oral
glucose intolerance.

| | | | | Direction of |
|---|---|---|---|---|
| Marker | Lower Limit | Upper Limit | Units | Association |
| AC6:0 | 0.44 | 0.70 | nMole per | Positive |
| | | | gram | |
| AC8:0 | 0.12 | 0.26 | nMole per | Positive |
| | | | gram | |
| AC10:0 | 0.12 | 0.32 | nMole per | Positive |
| | | | gram | |
| PE20:4n6 | 21.30 | 24.15 | Mole percent | Positive |
| PC18:0 | 12.40 | 14.20 | Mole percent | Positive |
| TG14:0 | 0.07 | 0.04 | Mole percent | Negative |

In addition to lipid metabolites that were independently predictive of AUC glucose and predictive of glucose intolerance as determined by 2-hour glucose, many lipid metabolites provided information that improved the power of fasted glucose to predict AUC glucose. Linear regression was employed to examine the correlation of fasting lipid metabolites with AUC glucose, adjusting for fasting glucose measurements. These fasted plasma lipid metabolites are shown in Table 7.

TABLE 7
Fasted Lipid Metabolites that Significantly Improve the Prediction of AUC
Glucose from a Fasted Glucose Measure.

| | | | Correlation | | Linear | | | | | |
|---|---|---|---|---|---|---|---|---|---|---|
| | | | with | | Regression: | | | | | |
| | | | Incremental | | Baseline | | | | | |
| | Correlation | | AUC | | Glucose + | | Correlation | | Correlation | |
| | with AUC | | Glucose | | Fatty Acid | | with Fasting | | with Fasting | |
| | Glucose (No | | (No | | Predicting | | Glucose (No | | Insulin (No | |
| | Diabetics) | | Diabetics) | | AUC Glucose | | Diabetics) | | Diabetics) | |
| | | P | | P | Baseline | | | P | | P |
| Predictors | R² | value | R² | value | Glc. | Analyte | R² | value | R² | value |
| PE20:4n6 | 0.52 | 0.00 | 0.24 | 0.17 | 0.00 | 0.00 | 0.09 | 0.62 | −0.08 | 0.64 |
| TG14:0 | −0.38 | 0.03 | −0.12 | 0.51 | 0.00 | 0.01 | 0.10 | 0.59 | 0.25 | 0.16 |
| TG14:1n5 | −0.25 | 0.17 | −0.21 | 0.24 | 0.00 | 0.01 | 0.30 | 0.09 | 0.32 | 0.07 |
| PC20:4n6 | 0.26 | 0.13 | 0.32 | 0.06 | 0.00 | 0.02 | −0.21 | 0.24 | −0.22 | 0.21 |
| FA20:5n3 | −0.18 | 0.31 | −0.28 | 0.12 | 0.00 | 0.02 | 0.33 | 0.06 | 0.12 | 0.52 |
| PE20:0 | −0.33 | 0.06 | −0.14 | 0.43 | 0.00 | 0.02 | 0.03 | 0.85 | −0.09 | 0.60 |
| FA22:4n6 | −0.17 | 0.33 | −0.19 | 0.27 | 0.00 | 0.05 | 0.26 | 0.13 | 0.03 | 0.88 |
| CE20:0 | 0.29 | 0.12 | 0.14 | 0.47 | 0.00 | 0.05 | −0.03 | 0.86 | 0.07 | 0.72 |
| CE14:0 | 0.23 | 0.20 | 0.23 | 0.20 | 0.00 | 0.05 | −0.14 | 0.43 | 0.01 | 0.96 |
| FA18:1n9 | 0.23 | 0.20 | −0.02 | 0.90 | 0.00 | 0.05 | −0.14 | 0.42 | −0.23 | 0.20 |
| LY22:4n6 | 0.11 | 0.55 | 0.17 | 0.35 | 0.00 | 0.06 | −0.35 | 0.05 | −0.31 | 0.08 |
| PC18:2n6 | −0.33 | 0.06 | −0.20 | 0.26 | 0.01 | 0.06 | −0.07 | 0.68 | −0.13 | 0.46 |
| FA24:1n9 | −0.23 | 0.19 | −0.13 | 0.48 | 0.00 | 0.06 | 0.10 | 0.56 | 0.08 | 0.67 |
| TG20:0 | −0.30 | 0.10 | −0.13 | 0.46 | 0.00 | 0.06 | −0.01 | 0.94 | −0.10 | 0.57 |
| FA22:2n6 | 0.49 | 0.01 | −0.01 | 0.95 | 0.03 | 0.07 | 0.41 | 0.03 | 0.40 | 0.04 |
| FA14:0 | −0.21 | 0.23 | −0.02 | 0.90 | 0.00 | 0.07 | 0.14 | 0.42 | 0.27 | 0.12 |
| TG20:2n6 | 0.15 | 0.40 | 0.19 | 0.29 | 0.00 | 0.09 | −0.23 | 0.20 | −0.13 | 0.46 |
| PC22:4n6 | 0.12 | 0.50 | 0.20 | 0.26 | 0.00 | 0.09 | −0.28 | 0.10 | −0.16 | 0.37 |
| CETotal.LC | 0.39 | 0.03 | 0.16 | 0.38 | 0.02 | 0.09 | 0.27 | 0.13 | 0.25 | 0.16 |
| FA18:1n7 | 0.15 | 0.39 | 0.10 | 0.57 | 0.00 | 0.09 | −0.22 | 0.21 | −0.06 | 0.73 |
| PE20:5n3 | −0.14 | 0.44 | −0.29 | 0.10 | 0.00 | 0.09 | 0.24 | 0.17 | 0.16 | 0.35 |
| PC18:0 | 0.34 | 0.05 | 0.04 | 0.81 | 0.01 | 0.10 | 0.19 | 0.29 | 0.35 | 0.04 |

Additionally, each metabolite was examined for its ability to predict glucose intolerant subjects by evaluating the AUC of a receiver-operator curve. For those metabolites that performed well, logistic regression was used to create a measure that combined fasting glucose with the metabolite. Improvements in the prediction of glucose intolerance using the combined variables were determined using AUC of the ROC curve (Table 8).

TABLE 8
Metabolites That Improve the Prediction of Glucose Intolerance
by a Fasted Glucose Measure as Assessed by Increased
AUC of an ROC Analysis.

| | |
|---|---|
| | Difference (AUC) After |
| AUC from ROC | Including Fasted |

| Predictor | Lipid + FG | Lipid | Glucose |
|---|---|---|---|
| FA20:5n3 | 0.76 | 0.65 | 0.11 |
| LY16:1n7 | 0.69 | 0.60 | 0.09 |
| PC20:2n6 | 0.71 | 0.63 | 0.08 |
| LY18:1n9 | 0.66 | 0.59 | 0.07 |
| FA22:6n3 | 0.65 | 0.59 | 0.07 |
| TG16:1n7 | 0.64 | 0.59 | 0.05 |
| PC20:1n9 | 0.70 | 0.66 | 0.04 |
| PC20:4n6 | 0.65 | 0.61 | 0.04 |
| TG22:4n6 | 0.62 | 0.59 | 0.03 |
| TG14:0 | 0.82 | 0.79 | 0.03 |
| PE20:4n6 | 0.71 | 0.69 | 0.03 |
| LY20:3n9 | 0.63 | 0.61 | 0.02 |
| TG14:1n5 | 0.74 | 0.72 | 0.02 |
| TG20:0 | 0.73 | 0.71 | 0.02 |
| FSTotal.LC | 0.71 | 0.69 | 0.02 |
| DG20:3n6 | 0.61 | 0.60 | 0.01 |
| PCdm18:1n9 | 0.60 | 0.59 | 0.01 |
| PC18:2n6 | 0.60 | 0.59 | 0.01 |
| PCdm18:0 | 0.60 | 0.59 | 0.01 |
| PE16:0 | 0.60 | 0.60 | 0.01 |
| DG18:1n7 | 0.65 | 0.65 | 0.01 |

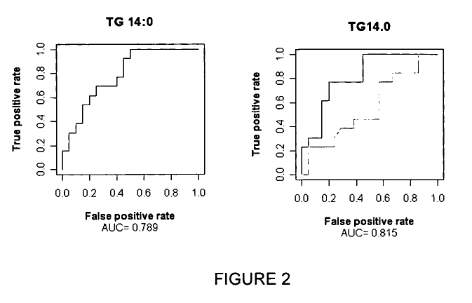

An example of the ability of lipid metabolites to improve the prediction of glucose tolerance by fasted glucose measured is shown in FIG. 2. TG14:0 performed better than fasting glucose (AUC 0.789) in the prediction of glucose intolerance. However, the combination of TG14:0 with fasting glucose resulted in an AUC of 0.815, which was an improvement over just TG14:0 or fasted glucose alone.

Fasted plasma metabolite concentrations that improve the prediction of glucose intolerance from fasted glucose measurement: CE14:0, CE20:0, CETotal:LC, DG18:1n7, DG20:3n6, FA14:0, FA18:1n7, FA18:1n9, FA20:5n3, FA22:6n3, FSTotal:LC, LY16:1n7, LY18:1n9, LY20:3n9, LY22:4n6, PC18:0, PC18:2n6, PC20:1n9, PC20:2n6, PC20:4n6, PC22:4n6, PCdm18:0, PCdm18:1n9, PE16:0, PE20:0, PE20:4n6, PE20:5n3, TG14:0, TG14:1n5, TG16:1n7, TG20:0, TG20:2n6, and TG22:4n6.

The markers CETotal:LC and PC18:0 were found to be positively associated with glucose intolerance, while PC18:2n6 was found to be negatively associated with glucose intolerance.

### Summary

This study used AUC glucose and 2-hour glucose as endpoints against which to benchmark plasma lipid metabolites for their power in assessing diabetic conditions. We suggest that 2-hour glucose is a logical surrogate for many aspects of the diabetic condition including type 2 diabetes, oral glucose intolerance, insulin resistance, etc. thus the markers of AUC glucose identified here are also markers of other diabetic conditions. The lipid metabolite markers described here may serve as stand alone predictive or diagnostic tests for aspects of the diabetic condition. Additionally, they may provide added value to existing or future tests including fasted glucose, fasted insulin, BMI, fasted triglycerides, LDL-cholesterol and other measures of metabolic status in the prediction of diabetic conditions.

### Example 2

### Markers of PPARs-Gamma Agonist Treatment Provide Diagnostic Tools for Treatment Efficacy and the Reversal of Diabetic Conditions

### Rationale

As demonstrated above, several plasma lipid metabolites provide better prediction of AUC glucose than fasted glucose and others add predictive value to fasted glucose measurements. Thus, lipid metabolism is playing an important role in glucose intolerance and other diabetic conditions. This fact is underscored by the fact that clinical treatment protocols for diabetic conditions employ drugs including thiazolidinediones, fibrates and statins that have profound effects on lipid metabolism. In particular, the group of agents known as PPARs agonists improve insulin resistance and diabetic conditions by altering lipid metabolism pathways and thus may produce changes in plasma lipid metabolite concentrations that will be useful as diagnostic tools for monitoring the efficacy of PPARs agonists in reversing diabetic conditions.

There are three PPARs receptors that are targeted by drug therapies, PPARs-alpha, PPARs-delta and PPARs-gamma. We profiled the changes in plasma lipid metabolite concentrations resulting from the treatment of human subjects with an example drug from each class to determine markers of treatment and efficacy. These markers may be useful in predicting the efficacy and safety of these drug classes in treating diabetic conditions. Further more, the markers identified here may help guide therapy choices (what drug, what dose, etc) by providing mechanistic diagnostic tools for diabetic conditions and their management by drug therapy.

### Study Overview

The primary objective of this exploratory research study was to describe the pharmacodynamic effect of placebo and rosiglitazone after 8 weeks of treatment and in the context of attendant glycemic changes. Males, aged 35-70 years with stable type 2 diabetes who are treated with diet and exercise alone, approved monotherapy, or approved low-dose combination therapy were be enrolled. After completion of a Screening Phase of up to 4 weeks and a 5-week Washout Phase, eligible subjects were be randomized to the placebo (20 subjects) or the rosiglitazone group (21 subjects). Treatments were single-blind for 8 weeks—and rosiglitazone dosages were titrated to achieve optimal glucose control (between 4-8 mg per day). Fasted plasma samples were taken at baseline and 4 and 8 weeks after initiating therapy.

Lipids measured in each sample included acylcarnitines (AC), butyrobetaine, L-carnitine, cholesterol, cholesterol esters (CE), diglycerides (DG), free cholesterol (FS), free fatty acids (FA), lysophosphatidylcholine (LY), phosphatidylcholine (PC), phosphatidylethanolamine (PE) and triglycerides (TG). For CE, DG, FA, LY, PC, PE and TG lipid classes the following fatty acid components were quantified as a proportion of total fatty acids within the lipid class: 14:0, 15:0, 16:0, 18:0, 20:0, 22:0, 24:0, 14:1n5, 16:1n7, 18:1n7, 18:1n9, 20:1n9, 20:3n9, 22:1n9, 24:1n9, 18:2n6, 18:3n6, 20:2n6, 20:3n6, 20:4n6, 22:2n6, 22:4n6, 22:5n6, 18:3n3, 18:4n3, 20:3n3, 20:4n3, 20:5n3, 22:5n3, 22:6n3, 24:6n3, plasmalogen derivatives of 16:0, 18:0, 18:1n7 and 18:1n9, t16:1n7 t18:1n9 t18:2n6. For the AC lipid class, the following fatty acid-carnitine esters were quantified in absolute terms (nMoles per gram of plasma or serum): 2:0, 3:0, 4:0, 5:0, 6:0, 8:0, 10:0, 12:0, 14:0, 16:0, 18:0, 18:1n9, 18:2n6. The term “Total.LC” indicates the value shown is the total concentration of the lipid class expressed as nMoles per gram of serum or plasma. Thus, the abbreviation PC18:2n6 indicates the percentage of plasma or serum phosphatidylcholine comprised of linoleic acid (18:2n6), the term AC6:0 indicates the absolute amount of hexanoylcarnitine present in serum or plasma and the term TGTotal.LC indicates the absolute amount of triglyceride present in plasma or serum.

### Results

Treatment with the PPARs-gamma agonists produced strong effects on the lipid metabolite concentrations in plasma. These changes provided diagnostic capabilities for assessing the efficacy of PPARs-gamma agents in treating diabetic conditions and in evaluating the safety and tolerability of treatment in individuals. Treatment induced improvements in metabolic parameters including reduced plasma glucose and HbA1c concentrations, and thus serves as potentially effective treatment for diabetic conditions.

The changes in plasma lipid metabolite concentrations induced by PPARs-gamma treatment may be directly related to mechanisms that reverse the diagnostic markers of diabetic conditions described above, or may be indicative of alternative pathways that improve diabetic conditions. In either case, the markers described below are useful in determining the efficacy of PPARs-gamma treatment in reversing or preventing diabetic conditions.

Significant changes (calculated by a paired t test of pre and post drug treatment for each treatment group) in the concentrations of plasma lipid metabolites induced by treatment are described in the lists below.

Plasma metabolites concentrations increased as a result of PPARs-gamma treatment included:

PC20:4n3, PC16:1n7, CE16:1n7, CE18:1n9, LY20:3n6, PC18:1n9, CE20:2n6, FA24:0, PE20:3n9, CE20:3n9, PC20:3n9, PE20:3n6, LY18:1n7, TG16:1n7, FA14:0, FA16:1n7, FA22:6n3, FA20:5n3, PC20:2n6, CETotal.LC, TG16:0, PC20:3n6, PE18:1n7, PE18:2n6, CE18:0, PE16:1n7, CE18:1n7, PE16:0, LY20:3n9, PC18:1n7, LY20:1n9, CE14:0, FA18:1n7, TG14:0, PC20:1n9, CE20:3n6, TG18:1n7, LY18:1n9, LY16:0, PC16:0, DGTotal.LC, DG16:0, DG18:0, LYTotal.LC, PETotal.LC

Plasma metabolite concentrations decreased as a result of PPARs-gamma treatment included:

PC20:4n6, CE20:4n6, TG22:4n6, PC20:0, LY22:5n3, FA18:1n9, DG18:1n9, LY20:5n3, PC22:6n3, FATotal.LC, TG22:6n3, PE20:4n6, LY18:0, PC18:0, FA22:5n3, CE18:2n6, LY20:4n6, FA18:2n6, LY18:2n6, DG18:2n6, PC18:4n3, LY18:3n3, TG20:5n3, DG20:4n6, TG20:4n6, PC18:3n3, TG18:3n3, Pedm, TG18:4n3, TG18:2n6, PCdm16:0, PEdm18:0, PEdm18:1n9, PC14:0, TG22:0, TG18:3n6, CE16:0, SP18:0

Selected plasma metabolites concentrations that increase as a result of PPARs-gamma treatment include:
The mole percent composition of 14:0 in TG and/or total plasma lipids;
The mole percent composition of 16:0 in PC, TG and/or total plasma lipids;
The mole percent composition of 16:1n7 in PC, CE, TG, FA and/or total plasma lipids;
The mole percent composition of 18:1n7 in PC, CE, TG, FA and/or total plasma lipids;
The mole percent composition of 18:1n9 in PC, CE and/or total plasma lipids;
The mole percent composition of 20:3n9 in PC, CE, TG and/or total plasma lipids; and/or
The mole percent composition of 20:3n6 in PC, CE and/or total plasma lipids.

Selected plasma metabolites concentrations that decrease as a result of PPARs-gamma treatment include:
The mole percent composition of 20:4n6 in PC, CE, TG and/or total plasma lipids;
The mole percent composition of 18:1n9 in FA;
The mole percent composition of 22:6n3 in PC, CE, TG and/or total plasma lipids;
The mole percent composition of 18:0 in PC and/or total plasma lipids;
The mole percent composition of 18:2n6 in PC, CE, FA TG and/or total plasma lipids;
The mole percent composition of plasmalogens (dm) in PC, PE and/or total plasma lipids; and/or
The mole percent composition of 20:3n6 in PC, CE and/or total plasma lipids.

### Example 3

### Markers of PPARs-Alpha and Delta Agonist Treatment Provide Diagnostic Tools for Treatment Efficacy, Safety and the Reversal of Diabetic Conditions

### Study Overview

A clinical study examining the effect of 12-week treatment with a PPARs-delta modifying agent (5 mg/10 mg) and PPARs-delta modifying agent (20 mg) as compared to placebo was performed in 57 subjects. Plasma samples were obtained at pre-dose and after 28, 42 and 84 days of treatment. Lipomics determined the concentration of lipid metabolites from each time point in the trial and evaluated the changes in lipid metabolite concentrations for markers of treatment efficacy, safety and the reversal of the diabetic condition.

Lipids measured included cholesterol, cholesterol esters (CE), diglycerides (DG), free cholesterol (FS), free fatty acids (FA), lysophosphatidylcholine (LY), phosphatidylcholine (PC), phosphatidylethanolamine (PE) and triglycerides (TG). For CE, DG, FA, LY, PC, PE and TG lipid classes the following fatty acid components were quantified as a proportion of total fatty acids within the lipid class: 14:0, 15:0, 16:0, 18:0, 20:0, 22:0, 24:0, 14:1n5, 16:1n7, 18:1n7, 18:1n9, 20:1n9, 20:3n9, 22:1n9, 24:1n9, 18:2n6, 18:3n6, 20:2n6, 20:3n6, 20:4n6, 22:2n6, 22:4n6, 22:5n6, 18:3n3, 18:4n3, 20:3n3, 20:4n3, 20:5n3, 22:5n3, 22:6n3, 24:6n3, plasmalogen derivatives of 16:0, 18:0, 18:1n7 and 18:1n9, t16:1n7 t18:1n9 t18:2n6. For the AC lipid class, the following fatty acid-carnitine esters were quantified in absolute terms (nMoles per gram of plasma or serum): 2:0, 3:0, 4:0, 5:0, 6:0, 8:0, 10:0, 12:0, 14:0, 16:0, 18:0, 18:1n9, 18:2n6. The term “Total.LC” indicates the value shown is the total concentration of the lipid class expressed as nMoles per gram of serum or plasma. Thus, the abbreviation PC18:2n6 indicates the percentage of plasma or serum phosphatidylcholine comprised of linoleic acid (18:2n6), the term AC6:0 indicates the absolute amount of hexanoylcarnitine present in serum or plasma and the term TGTotal.LC indicates the absolute amount of triglyceride present in plasma or serum.

### Results

Treatment with both PPARs-a and PPARs-d agonists produced strong effects on the lipid metabolite concentrations in plasma. These changes provided diagnostic capabilities for assessing the efficacy of PPARs agents in treating diabetic conditions and in evaluating the safety and tolerability of treatment in individuals. Both treatments provided improvements in metabolic parameters including plasma triglyceride, LDL and glucose levels, and thus serve as potentially effective treatments for diabetic conditions.

The changes in plasma lipid metabolite concentrations induced by PPARs treatment may be directly related to mechanisms that reverse the diagnostic markers of diabetic conditions described above, or may be indicative of alternative pathways that improve diabetic conditions. In either case, the markers described below are useful in determining the efficacy of PPARs treatment in reversing or preventing diabetic conditions.

Significant changes in the concentrations of plasma lipid metabolites induced by treatment are described in the lists below. The change from day 1 to day 28, 42, and 84 was calculated for each treatment group and significance was assessed by comparing the change within the treatment group to that of the placebo group using unpaired t-tests.

Plasma metabolites concentrations increased as a result of PPARs-alpha treatment included:

CE16:1n7, CE18:1n9, CE18:3n6, CE20:3n9, CE20:4n6, DG14:0, DG14:1n5, DG15:0, DG16:0, DG18:0, DG20:4n6, DG22:6n3, DG24:0, FA14:1n5, FA15:0, FA16:0, FA18:0, FA20:0, FA22:0, FA22:1n9, FA24:0, FA24:1n9, LY16:0, LY18:3n6, LY20:4n3, PC16:0, PC16:1n7, PC18:1n9, PC18:3n6, PC18:4n3, PC20:2n6, PC20:3n6, PC20:3n9, PC20:4n3, PCdm16:0, PCdm18:1n7, PE16:1n7, PEdm16:0, PEdm18:1n7, TG15:0, TG16:0, TG16:1n7, TG20:3n9, TG20:4n6, TG22:4n6, TG22:5n6, TG24:0, TG18.3n6, TG18.4n3

Plasma metabolites concentrations decreased as a result of PPARs-alpha treatment included:

CE18:2n6, CETotal.LC, DG18:1n7, DG18:1n9, DG18:2n6, DGTotal.LC, FA18:1n9, FA18:2n6, FA20:1n9, FATotal.LC, PC18:2n6, PC22:5n3, PE18:0, PE22:0, PE22:1n9, TG18:2n6, TG18:3n3, TGTotal.LC

Plasma metabolite concentrations increased as a result of PPARs-delta treatment included:

CE16:1n7, CE18:1n9, CE18:3n6, CE20:3n9, DG14:0, DG15:0, DG16:0, DG16:1n7, FA14:0, FA14:1n5, FA15:0, FA18:0, FA20:0, FA20:4n6, FA22:0, FA22:2n6, FA22:5n6, FA24:1n9, LY16:1n7, LY18:1n9, LY18:3n6, LY20:3n9, PC16:1n7, PC18:1n9, PC18:3n3, PC18:3n6, PC20:2n6, PC20:3n9, PC20:4n3, PC20:5n3, PCdm16:0, PCdm18:1n9, PE16:1n7, PE18:1n7, PE20:3n9, TG14:0, TG14:1n5, TG16:0, TG16:1n7, TG18:3n6, TG18:4n3, TG20:3n9, TG20:4n6, TG22:4n6, TG24:1n9, L-carnitine and butyrobetaine.

Plasma metabolite concentrations decreased as a result of PPARs-delta treatment included:

CE18:1n7, CE18:2n6, CE20:4n6, CE22:1n9, CETotal.LC, DG18:2n6, FA18:1n7, FA18:1n9, FA20:1n9, FA22:6n3, FATotal.LC, LY18:0, LY20:4n6, LY22:6n3, PC15:0, PC20:4n6, PC22:5n6, PC22:6n3, PE18:0, PE22:6n3, TG18:2n6, TG18:3n3, CE16:0, DG18:3n3, DG20:3n6, DGTotal.LC, FA18:2n6, FA20:2n6, FA20:3n6, PC18:2n6, PE20:2n6, PEdm18:0, PETotal.LC and TGTotal.LC

Selected plasma metabolites concentrations that increase as a result of PPARs-alpha or delta treatment include:
The mole percent composition of 16:0 in PC, TG and/or total plasma lipids;
The mole percent composition of 16:1n7 in PC, CE, TG, FA and/or total plasma lipids;
The mole percent composition of 18:1n9 in PC, CE and/or total plasma lipids;
The mole percent composition of 20:3n9 in PC, CE, TG and/or total plasma lipids;
The mole percent composition of 18:3n6 in PC, CE, TG and/or total plasma lipids;
The mole percent composition of 20:3n6 in PC, CE and/or total plasma lipids; and/or
The mole percent composition of plasmalogens (dm) in PC, PE and/or total plasma lipids.

Selected plasma metabolites concentrations that decrease as a result of PPARs-alpha or delta treatment include:
The mole percent composition of 18:1n9 in FA;
The mole percent composition of 22:6n3 in PC, CE, TG and/or total plasma lipids; and/or
The mole percent composition of 18:2n6 in PC, CE, FA TG and/or total plasma lipids.

### Example 4

### Markers of Response to Therapy

Drugs targeting all three PPARs targets (PPARs-gamma, PPARs-alpha, and PPARs-delta) are in use or have been potential use as anti-diabetic agents. The following lists combine the common markers of PPARs treatment to identify markers of therapeutic response. These markers can be used to assess the efficacy of the treatment of diabetic conditions with PPARs agents:

These metabolites increase with therapeutic improvements in oral glucose intolerance, insulin resistance and other diabetic conditions:
The mole percent composition of 14:0 in TG and/or total plasma lipids;
The mole percent composition of 16:0 in PC, TG and/or total plasma lipids;
The mole percent composition of 16:1n7 in PC, CE, TG, FA and/or total plasma lipids;
The mole percent composition of 18:1n9 in PC, CE and/or total plasma lipids;
The mole percent composition of 20:3n9 in PC, CE, TG and/or total plasma lipids; and/or
The mole percent composition of 20:3n6 in PC, CE and/or total plasma lipids.

These metabolites decrease with therapeutic improvements in oral glucose intolerance, insulin resistance and other diabetic conditions:
The mole percent composition of 18:1n9 in FA;
The mole percent composition of 22:6n3 in PC, CE, TG and/or total plasma lipids;
The mole percent composition of 18:0 in PC and/or total plasma lipids;
The mole percent composition of 18:2n6 in PC, CE, FA TG and/or total plasma lipids;
The mole percent composition of 18:3n6 in PC, CE, TG and/or total plasma lipids; and/or
The mole percent composition of 20:3n6 in PC, CE and/or total plasma lipids.

### REFERENCES

References cited above with numerical notation in endnote form are identified below:
1. Knowler, W. C., et al., Reduction in the incidence of type 2 diabetes with lifestyle intervention or metformin. N Engl J Med, 2002. 346(6): p. 393-403.
2. Kahn, S. E., The relative contributions of insulin resistance and beta-cell dysfunction to the pathophysiology of Type 2 diabetes. Diabetologia, 2003. 46(1): p. 3-19.
3. Chiasson, J. L. and R. Rabasa-Lhoret, Prevention of type 2 diabetes: insulin resistance and Beta-cell function. Diabetes, 2004. 53 Suppl 3: p. S34-8.
4. Paolisso, G., et al., A high concentration of fasting plasma non-esterified fatty acids is a risk factor for the development of NIDDM. Diabetologia, 1995. 38(10): p. 1213-7.
5. Charles, M. A., et al., The role of non-esterified fatty acids in the deterioration of glucose tolerance in Caucasian subjects: results of the Paris Prospective Study. Diabetologia, 1997. 40(9): p. 1101-6.
6. Bays, H., L. Mandarin, and R. A. DeFronzo, Role of the adipocyte, free fatty acids, and ectopic fat in pathogenesis of type 2 diabetes mellitus: peroxisomal proliferator-activated receptor agonists provide a rational therapeutic approach. J Clin Endocrinol Metab, 2004. 89(2): p. 463-78.
7. Faraj, M., H. L. Lu, and K. Cianflone, Diabetes, lipids, and adipocyte secretagogues. Biochem Cell Biol, 2004. 82(1): p. 170-90.
8. Kahn, B. B. and J. S. Flier, Obesity and insulin resistance. J Clin Invest, 2000. 106(4): p. 473-81.
9. Greenberg, A. S. and M. L. McDaniel, Identifying the links between obesity, insulin resistance and beta-cell function: potential role of adipocyte-derived cytokines in the pathogenesis of type 2 diabetes. Eur J Clin Invest, 2002. 32 Suppl 3: p. 24-34.
10. Dandona, P., A. Aljada, and A. Bandyopadhyay, Inflammation: the link between insulin resistance, obesity and diabetes. Trends Immunol, 2004. 25(1): p. 4-7.
11. Lam, T. K., G. van de Werve, and A. Giacca, Free fatty acids increase basal hepatic glucose production and induce hepatic insulin resistance at different sites. Am J Physiol Endocrinol Metab, 2003. 284(2): p. E281-90.
12. Park, J., et al., Chronic exogenous insulin and chronic carbohydrate supplementation increase de novo VLDL triglyceride fatty acid production in rats. J Lipid Res, 1997. 38(12): p. 2529-36.
13. Aarsland, A., D. Chinkes, and R. R. Wolfe, Contributions of de novo synthesis of fatty acids to total VLDL-triglyceride secretion during prolonged hyperglycemia/hyperinsulinemia in normal man. J Clin Invest, 1996. 98(9): p. 2008-17.
14. Adeli, K., et al., Mechanisms of hepatic very low-density lipoprotein overproduction in insulin resistance. Trends Cardiovasc Med, 2001. 11(5): p. 170-6.
15. Taghibiglou, C., et al., Mechanisms of hepatic very low density lipoprotein overproduction in insulin resistance. Evidence for enhanced lipoprotein assembly, reduced intracellular ApoB degradation, and increased microsomal triglyceride transfer protein in a fructose-fed hamster model. J Biol Chem, 2000. 275(12): p. 8416-25.
16. Krssak, M., et al., Intramyocellular lipid concentrations are correlated with insulin sensitivity in humans: a 1H NMR spectroscopy study. Diabetologia, 1999. 42(1): p. 113-6.
17. Petersen, K. F. and G. I. Shulman, Pathogenesis of skeletal muscle insulin resistance in type 2 diabetes mellitus. Am J Cardiol, 2002. 90(5A): p. 11G-18G.
18. Shulman, G. I., Cellular mechanisms of insulin resistance in humans. Am J Cardiol, 1999. 84(1A): p. 3J-10J.
19. Shulman, G. I., Cellular mechanisms of insulin resistance. J Clin Invest, 2000. 106(2): p. 171-6.
20. Petersen, K. F., et al., Impaired mitochondrial activity in the insulin-resistant offspring of patients with type 2 diabetes. N Engl J Med, 2004. 350(7): p. 664-71.
21. Hulver, M. W., et al., Skeletal muscle lipid metabolism with obesity. Am J Physiol Endocrinol Metab, 2003. 284(4): p. E741-7.
22. Polonsky, K. S., J. Sturis, and G. I. Bell, Seminars in Medicine of the Beth Israel Hospital, Boston. Non-insulin-dependent diabetes mellitus—a genetically programmed failure of the beta cell to compensate for insulin resistance. N Engl J Med, 1996. 334(12): p. 777-83.
23. Weir, G. C., Which comes first in non-insulin-dependent diabetes mellitus: insulin resistance or beta-cell failure? Both come first. Jama, 1995. 273(23): p. 1878-9.
24. Mauvais-Jarvis, F. and C. R. Kahn, Understanding the pathogenesis and treatment of insulin resistance and type 2 diabetes mellitus: what can we learn from transgenic and knockout mice? Diabetes Metab, 2000. 26(6): p. 433-48.
25. Mauvais-Jarvis, F., et al., A model to explore the interaction between muscle insulin resistance and beta-cell dysfunction in the development of type 2 diabetes. Diabetes, 2000. 49(12): p. 2126-34.
26. Weir, G. C. and S. Bonner-Weir, Five stages of evolving Beta-cell dysfunction during progression to diabetes. Diabetes, 2004. 53 Suppl 3: p. S16-21.
27. Biden, T. J., et al., Chronic effects of fatty acids on pancreatic beta-cell function: new insights from functional genomics. Diabetes, 2004. 53 Suppl 1: p. S159-65.
28. Manco, M., M. Calvani, and G. Mingrone, Effects of dietary fatty acids on insulin sensitivity and secretion. Diabetes Obes Metab, 2004. 6(6): p. 402-13.
29. Mason, T. M., et al., Prolonged elevation of plasma free fatty acids desensitizes the insulin secretory response to glucose in vivo in rats. Diabetes, 1999. 48(3): p. 524-30.
30. El-Assaad, W., et al., Saturated fatty acids synergize with elevated glucose to cause pancreatic beta-cell death. Endocrinology, 2003. 144(9): p. 4154-63.
31. Lewis, G. F., et al., Disordered fat storage and mobilization in the pathogenesis of insulin resistance and type 2 diabetes. Endocr Rev, 2002. 23(2): p. 201-29.
32. Rabasa-Lhoret, R., et al., Modified quantitative insulin sensitivity check index is better correlated to hyperinsulinemic glucose clamp than other fasting-based index of insulin sensitivity in different insulin-resistant states. J Clin Endocrinol Metab, 2003. 88(10): p. 4917-23.
33. Vessby, B., et al., The risk to develop NIDDM is related to the fatty acid composition of the serum cholesterol esters. Diabetes, 1994. 43(11): p. 1353-7.
34. Salomaa, V., et al., Fatty acid composition of serum cholesterol esters in different degrees of glucose intolerance: a population-based study. Metabolism, 1990. 39(12): p. 1285-91.
35. Wang, L., et al., Plasma fatty acid composition and incidence of diabetes in middle-aged adults: the Atherosclerosis Risk in Communities (ARIC) Study. Am J Clin Nutr, 2003. 78(1): p. 91-8.
36. Lovejoy, J. C., et al., Relationship of dietary fat and serum cholesterol ester and phospholipid fatty acids to markers of insulin resistance in men and women with a range of glucose tolerance. Metabolism, 2001. 50(1): p. 86-92.
37. Vessby, B., S. Tengblad, and H. Lithell, Insulin sensitivity is related to the fatty acid composition of serum lipids and skeletal muscle phospholipids in 70-year-old men. Diabetologia, 1994. 37(10): p. 1044-50.
38. Ferre, P., The biology of peroxisome proliferator-activated receptors: relationship with lipid metabolism and insulin sensitivity. Diabetes, 2004. 53 Suppl 1: p. S43-50.
39. Berger, J. and D. E. Moller, The mechanisms of action of PPARs. Annu Rev Med, 2002. 53: p. 409-35.
40. Kota, B. P., T. H. Huang, and B. D. Roufogalis, An overview on biological mechanisms of PPARs. Pharmacol Res, 2005. 51(2): p. 85-94.
41. Brash, A. R., Lipoxygenases: occurrence, functions, catalysis, and acquisition of substrate. J Biol Chem, 1999. 274(34): p. 23679-82.
42. Needleman, P., et al., Arachidonic acid metabolism. Annu Rev Biochem, 1986. 55: p. 69-102.
43. Lawson, J. A., J. Rokach, and G. A. FitzGerald, Isoprostanes: formation, analysis and use as indices of lipid peroxidation in vivo. J Biol Chem, 1999. 274(35): p. 24441-4.
44. Sigauke, E., et al., Carnitine palmitoyltransferase II deficiency: a clinical, biochemical, and molecular review. Lab Invest, 2003. 83(11): p. 1543-54.
45. Fukao, T., G. D. Lopaschuk, and G. A. Mitchell, Pathways and control of ketone body metabolism: on the fringe of lipid biochemistry. Prostaglandins Leukot Essent Fatty Acids, 2004. 70(3): p. 243-51.
46. Hoppel, C. L. and S. M. Genuth, Urinary excretion of acetylcarnitine during human diabetic and fasting ketosis. Am J Physiol, 1982. 243(2): p. E168-72.
47. Hoppel, C. L. and S. M. Genuth, Carnitine metabolism in normal-weight and obese human subjects during fasting. Am J Physiol, 1980. 238(5): p. E409-15.
48. Mingrone, G., Carnitine in type 2 diabetes. Ann N Y Acad Sci, 2004. 1033: p. 99-107.
49. Frohlich, J., et al., Effect of fasting on free and esterified carnitine levels in human serum and urine: correlation with serum levels of free fatty acids and beta-hydroxybutyrate. Metabolism, 1978. 27(5): p. 555-61.
50. Inokuchi, T., et al., Changes in carnitine metabolism with ketone body production in obese glucose-intolerant patients. Diabetes Res Clin Pract, 1995. 30(1): p. 1-7.
51. Su, X., et al., Accumulation of long-chain acylcarnitine and 3-hydroxy acylcarnitine molecular species in diabetic myocardium: identification of alterations in mitochondrial fatty acid processing in diabetic myocardium by shotgun lipidomics. Biochemistry, 2005. 44(13): p. 5234-45.
52. Cree, M. G., et al., Intramuscular and liver triglycerides are increased in the elderly. J Clin Endocrinol Metab, 2004. 89(8): p. 3864-71.
53. Folch, J., M. Lees, and G. H. Sloane Stanley, A simple method for the isolation and purification of total lipides from animal tissues. J Biol Chem, 1957. 226(1): p. 497-509.
54. Watkins, S. M., et al., Unique phospholipid metabolism in mouse heart in response to dietary docosahexaenoic or alpha-linolenic acids. Lipids, 2001. 36(3): p. 247-54.
55. Thompson, W. G., E. J. Bergstralh, and J. M. Slezak, Use of glucose, insulin, and C-reactive protein to determine need for glucose tolerance testing. Obes Res, 2003. 11(8): p. 1027-32.

1-6. (canceled)

7. A method for assessing a diabetic condition in a subject comprising measuring the levels of at least one of a first, second, third, and fourth metabolite marker in a sample from the subject wherein the first, second, third, and fourth metabolite markers are selected from the group consisting of AC6:0, PE20:4n6, AC16:0, AC14:0, FA22:2n6, AC8:0, AC10:0, AC3:0, CETotal:LC, AC12:0, TG14:0, TGTotal:LC, PE16:1n7, PC18:0, L-Carnitine, PE20:0, PC18:2n6, DGTotal:LC, AC4:0, TG18:1n9, DG18:0, CE18:2n6, CE16:1n7, PC16:1n7, FA16:1n7, FA18:1n9, PE20:4n6, PC20:4n6, CE14:0, CETotal:LC, TG16:1n7, FSTotal:LC, PCdm, CE18:1n9, PC18:1n9, and PE16:0, and wherein the levels of the first, second, third, and fourth metabolite markers are indicative of the presence, absence, or degree of the diabetic condition.

8. The method of claim 7, which further comprises correlating the level of the first, second, third, and fourth metabolite markers with the presence, absence, or degree of the diabetic condition, wherein if the first, second, third, or fourth marker is AC6:0, PE20:4n6, AC16:0, AC14:0, FA22:2n6, AC8:0, AC10:0, AC3:0, CETotal:LC, AC12:0, TGTotal:LC, PC18:0, L-Carnitine, PE20:0, DGTotal:LC, AC4:0, TG18:1n9, PE20:4n6, PC20:4n6, CE14:0, CETotal:LC, FSTotal:LC, or PCdm, the first, second, third, or fourth marker is positively correlated with the presence, risk of developing, or severity of the diabetic condition, and wherein if the first, second, third, or fourth marker is TG14:0, PE16:1n7, PE20:0, PC18:2n6, DG18:0 and CE18:2n6, CE16:1n7, CE18:1n9, PC16:1n7, PC18:1n9, FA16:1n7, FA18:1n9, TG16:1n7, or PE16:0, the first, second, third, or fourth marker is negatively correlated with the presence, risk of developing, or severity of the diabetic condition.

9. The method of claim 7, wherein at least one marker is selected from the group consisting of: AC6:0, AC16:0, AC14:0, AC8:0, AC10:0, AC12:0, TG14:0, FA16:1n7, FA18:1n9, CE16:1n7, PC16:1n7 and PC18:1n9.

10. The method of claim 7, wherein at least two markers are selected from the group consisting of: AC6:0, AC16:0, AC14:0, AC8:0, AC10:0, AC12:0, TG14:0, FA16:1n7, FA18:1n9, CE16:1n7, PC16:1n7 and PC18:1n9.

11. The method of claim 7, wherein at least three markers are selected from the group consisting of: AC6:0, AC16:0, AC14:0, AC8:0, AC10:0, AC12:0, TG14:0, FA16:1n7, FA18:1n9, CE16:1n7, PC16:1n7 and PC18:1n9.

12. The method of claim 7, wherein at least four markers are selected from the group consisting of: AC6:0, AC16:0, AC14:0, AC8:0, AC10:0, AC12:0, TG14:0, FA16:1n7, FA18:1n9, CE16:1n7, PC16:1n7 and PC18:1n9.

13. The method of claim 7, wherein the diabetic condition is impaired glucose tolerance, insulin resistance, hepatic steatosis, non-alcoholic steatohepatitis (NASH), pediatric NASH, obesity, childhood obesity, metabolic syndrome, polycystic ovary disease, or gestational diabetes.

14-18. (canceled)

19. The method of claim 7, which is a method of identifying, monitoring, or assessing the severity of the diabetic condition.

20. The method of claim 7, which is a method of assessing the progression or regression of the diabetic condition.

21. The method of claim 7, which further comprises:
(1) determining the presence or absence of one or more risk factors for the diabetic condition, and correlating the presence or absence of the risk factor with the presence, risk of developing, or severity of the diabetic condition; or
(2) measuring the level of an additional biomarker, and correlating the level of the additional biomarker with the presence, risk of developing, or severity of the diabetic condition.

22. The method of claim 21, wherein the one or more risk factors are selected from the group consisting of: age, weight, body mass index (BMI), family history, medical history, ethnic background, high blood pressure, cholesterol level, and activity level.

23. The method of claim 21, wherein the additional biomarker is selected from the group consisting of blood glucose or glycosylated hemoglobin.

24. A method of determining the characteristic of an agent comprising selecting a method from the group consisting of:
(a) administering the agent to a subject and measuring the level of a first and optionally a second metabolite marker selected from the group consisting of PC20:4n6, CE20:4n6, TG22:4n6, PC20:0, LY22:5n3, FA18:1n9, DG18:1n9, LY20:5n3, PC22:6n3, FATotal.LC, TG22:6n3, PE20:4n6, LY18:0, PC18:0, FA22:5n3, CE18:2n6, LY20:4n6, FA18:2n6, LY18:2n6, DG18:2n6, PC18:4n3, LY18:3n3, TG20:5n3, DG20:4n6, TG20:4n6, PC18:3n3, TG18:3n3, Pedm, TG18:4n3, TG18:2n6, PCdm16:0, PEdm18:0, PEdm18:1n9, PC14:0, TG22:0, TG18:3n6, CE16:0, SP18:0, wherein a decrease from the normal level of the metabolite marker is indicative that the agent affects PPAR-γ;
(b) administering the agent to a subject and measuring the level of a first and optionally a second metabolite marker selected from the group consisting of PC20:4n3, PC16:1n7, CE16:1n7, CE18:1n9, LY20:3n6, PC18:1n9, CE20:2n6, FA24:0, PE20:3n9, CE20:3n9, PC20:3n9, PE20:3n6, LY18:1n7, TG16:1n7, FA14:0, FA16:1n7, FA22:6n3, FA20:5n3, PC20:2n6, CETotal.LC, TG16:0, PC20:3n6, PE18:1n7, PE18:2n6, CE18:0, PE16:1n7, CE18:1n7, PE16:0, LY20:3n9, PC18:1n7, LY20:1n9, CE14:0, FA18:1n7, TG14:0, PC20:1n9, CE20:3n6, TG18:1n7, LY18:1n9, LY16:0, PC16:0, DGTotal.LC, DG16:0, DG18:0, LYTotal.LC, PETotal.LC wherein an increase from the normal level of the metabolite marker is indicative that the agent affects PPAR-γ;
(c) administering the agent to a subject and measuring the level of at least one first metabolite marker selected from the group consisting of PC20:4n6, CE20:4n6, TG22:4n6, PC20:0, LY22:5n3, FA18:1n9, DG18:1n9, LY20:5n3, PC22:6n3, FATotal.LC, TG22:6n3, PE20:4n6, LY18:0, PC18:0, FA22:5n3, CE18:2n6, LY20:4n6, FA18:2n6, LY18:2n6, DG18:2n6, PC18:4n3, LY18:3n3, TG20:5n3, DG20:4n6, TG20:4n6, PC18:3n3, TG18:3n3, Pedm, TG18:4n3, TG18:2n6, PCdm16:0, PEdm18:0, PEdm18:1n9, PC14:0, TG22:0, TG18:3n6, CE16:0, SP18:0, and the level of at least one second metabolite marker selected from the group consisting of PC20:4n3, PC16:1n7, CE16:1n7, CE18:1n9, LY20:3n6, PC18:1n9, CE20:2n6, FA24:0, PE20:3n9, CE20:3n9, PC20:3n9, PE20:3n6, LY18:1n7, TG16:1n7, FA14:0, FA16:1n7, FA22:6n3, FA20:5n3, PC20:2n6, CETotal.LC, TG16:0, PC20:3n6, PE18:1n7, PE18:2n6, CE18:0, PE16:1n7, CE18:1n7, PE16:0, LY20:3n9, PC18:1n7, LY20:1n9, CE14:0, FA18:1n7, TG14:0, PC20:1n9, CE20:3n6, TG18:1n7, LY18:1n9, LY16:0, PC16:0, DGTotal.LC, DG16:0, DG18:0, LYTotal.LC, PETotal.LC, wherein a decrease from the normal level of the first metabolite marker and an increase from the normal level of the second metabolite marker are indicative that the agent affects PPAR-γ;
(d) administering the agent to a subject and measuring the level of a first and optionally a second metabolite marker selected from the group consisting of CE18:2n6, CETotal.LC, DG18:1n7, DG18:1n9, DG18:2n6, DGTotal.LC, FA18:1n9, FA18:2n6, FA20:1n9, FATotal.LC, PC18:2n6, PC22:5n3, PE18:0, PE22:0, PE22:1n9, TG18:2n6, TG18:3n3, TGTotal.LC wherein a decrease from the normal level of the metabolite marker is indicative of an agent affecting PPAR-α;
(e) administering the agent to a subject and measuring the level of a first and optionally a second metabolite marker selected from the group consisting of CE16:1n7, CE18:1n9, CE18:3n6, CE20:3n9, CE20:4n6, DG14:0, DG14:1n5, DG15:0, DG16:0, DG18:0, DG20:4n6, DG22:6n3, DG24:0, FA14:1n5, FA15:0, FA16:0, FA18:0, FA20:0, FA22:0, FA22:1n9, FA24:0, FA24:1n9, LY16:0, LY18:3n6, LY20:4n3, PC16:0, PC16:1n7, PC18:1n9, PC18:3n6, PC18:4n3, PC20:2n6, PC20:3n6, PC20:3n9, PC20:4n3, PCdm16:0, PCdm18:1n7, PE16:1n7, PEdm16:0, PEdm18:1n7, TG15:0, TG16:0, TG16:1n7, TG20:3n9, TG20:4n6, TG22:4n6, TG22:5n6, TG24:0, TG18.3n6, TG18.4n3, wherein an increase from the normal level of the metabolite marker is indicative that the agent affects PPAR-α;
(f) administering the agent to a subject and measuring the level of at least one first metabolite marker selected from the group consisting of CE18:2n6, CETotal.LC, DG18:1n7, DG18:1n9, DG18:2n6, DGTotal.LC, FA18:1n9, FA18:2n6, FA20:1n9, FATotal.LC, PC18:2n6, PC22:5n3, PE18:0, PE22:0, PE22:1n9, TG18:2n6, TG18:3n3, TGTotal.LC and the level of at least one second metabolite marker selected from the group consisting of CE16:1n7, CE18:1n9, CE18:3n6, CE20:3n9, CE20:4n6, DG14:0, DG14:1n5, DG15:0, DG16:0, DG18:0, DG20:4n6, DG22:6n3, DG24:0, FA14:1n5, FA15:0, FA16:0, FA18:0, FA20:0, FA22:0, FA22:1n9, FA24:0, FA24:1n9, LY16:0, LY18:3n6, LY20:4n3, PC16:0, PC16:1n7, PC18:1n9, PC18:3n6, PC18:4n3, PC20:2n6, PC20:3n6, PC20:3n9, PC20:4n3, PCdm16:0, PCdm18:1n7, PE16:1n7, PEdm16:0, PEdm18:1n7, TG15:0, TG16:0, TG16:1n7, TG20:3n9, TG20:4n6, TG22:4n6, TG22:5n6, TG24:0, TG18.3n6, TG18.4n3 wherein a decrease from the normal level of the first metabolite marker and an increase from the normal level of the second metabolite marker are indicative that the agent affects PPAR-α;
(g) administering the agent to a subject and measuring the level of a first and optionally a second metabolite marker selected from the group consisting of CE18:1n7, CE18:2n6, CE20:4n6, CE22:1n9, CETotal.LC, DG18:2n6, FA18:1n7, FA18:1n9, FA20:1n9, FA22:6n3, FATotal.LC, LY18:0, LY20:4n6, LY22:6n3, PC15:0, PC20:4n6, PC22:5n6, PC22:6n3, PE18:0, PE22:6n3, TG18:2n6, TG18:3n3, CE16:0, DG18:3n3, DG20:3n6, DGTotal.LC, FA18:2n6, FA20:2n6, FA20:3n6, PC18:2n6, PE20:2n6, PEdm18:0, PETotal.LC and TGTotal.LC, wherein a decrease from the normal level of the metabolite marker is indicative that the agent affects PPAR-δ;
(h) administering the agent to a subject and measuring the level of a first and optionally a second metabolite marker selected from the group consisting of CE16:1n7, CE18:1n9, CE18:3n6, CE20:3n9, DG14:0, DG15:0, DG16:0, DG16:1n7, FA14:0, FA14:1n5, FA15:0, FA18:0, FA20:0, FA20:4n6, FA22:0, FA22:2n6, FA22:5n6, FA24:1n9, LY16:1n7, LY18:1n9, LY18:3n6, LY20:3n9, PC16:1n7, PC18:1n9, PC18:3n3, PC18:3n6, PC20:2n6, PC20:3n9, PC20:4n3, PC20:5n3, PCdm16:0, PCdm18:1n9, PE16:1n7, PE18:1n7, PE20:3n9, TG14:0, TG14:1n5, TG16:0, TG16:1n7, TG18:3n6, TG18:4n3, TG20:3n9, TG20:4n6, TG22:4n6, TG24:1n9, L-carnitine and butyrobetaine, wherein an increase from the normal level of the first metabolite marker is indicative that the agent affects PPAR-δ; and
(i) administering the agent to a subject and measuring the level of at least one first metabolite marker selected from the group consisting of CE18:1n7, CE18:2n6, CE20:4n6, CE22:1n9, CETotal.LC, DG18:2n6, FA18:1n7, FA18:1n9, FA20:1n9, FA22:6n3, FATotal.LC, LY18:0, LY20:4n6, LY22:6n3, PC15:0, PC20:4n6, PC22:5n6, PC22:6n3, PE18:0, PE22:6n3, TG18:2n6, TG18:3n3, CE16:0, DG18:3n3, DG20:3n6, DGTotal.LC, FA18:2n6, FA20:2n6, FA20:3n6, PC18:2n6, PE20:2n6, PEdm18:0, PETotal.LC and TGTotal.LC, and measuring the level of at least one second metabolite marker selected from the group consisting of CE16:1n7, CE18:1n9, CE18:3n6, CE20:3n9, DG14:0, DG15:0, DG16:0, DG16:1n7, FA14:0, FA14:1n5, FA15:0, FA18:0, FA20:0, FA20:4n6, FA22:0, FA22:2n6, FA22:5n6, FA24:1n9, LY16:1n7, LY18:1n9, LY18:3n6, LY20:3n9, PC16:1n7, PC18:1n9, PC18:3n3, PC18:3n6, PC20:2n6, PC20:3n9, PC20:4n3, PC20:5n3, PCdm16:0, PCdm18:1n9, PE16:1n7, PE18:1n7, PE20:3n9, TG14:0, TG14:1n5, TG16:0, TG16:1n7, TG18:3n6, TG18:4n3, TG20:3n9, TG20:4n6, TG22:4n6, TG24:1n9, L-carnitine and butyrobetaine, wherein a decrease from the normal level of the first metabolite marker and an increase from the normal level of the second metabolite marker are indicative that the agent affects PPAR-δ.

25-44. (canceled)

45. A method of assessing the response of a subject having a diabetic condition to a treatment for the diabetic condition, comprising measuring the level of a metabolite marker in a sample from the subject following administration of the treatment to the subject, wherein the one or more metabolite markers are selected from the group consisting of: the relative amount of 14:0 to total fatty acid content in TG; the relative amount of 14:0 to total fatty acid content in total lipids; the relative amount of 16:0 to total fatty acid content in PC; the relative amount of 16:0 to total fatty acid content in TG; the relative amount of 16:0 to total fatty acid content in total lipids; the relative amount of 16:1n7 to total fatty acid content in PC; the relative amount of 16:1n7 to total fatty acid content in CE; the relative amount of 16:1n7 to total fatty acid content in TG; the relative amount of 16:1n7 to total fatty acid content in FA; the relative amount of 16:1n7 to total fatty acid content in total lipids; the relative amount of 18:1n9 to total fatty acid content in PC; the relative amount of 18:1n9 to total fatty acid content in CE; the relative amount of 18:1n9 to total fatty acid content in total lipids; the relative amount of 20:3n9 to total fatty acid content in PC; the relative amount of 20:3n9 to total fatty acid content in CE; the relative amount of 20:3n9 to total fatty acid content in TG; the relative amount of 20:3n9 to total fatty acid content in total lipids; the relative amount of 20:3n6 to total fatty acid content in PC; the relative amount of 20:3n6 to total fatty acid content in CE; the relative amount of 20:3n6 to total fatty acid content in total lipids; the relative amount of 18:1n9 to total fatty acid content in FA; the relative amount of 22:6n3 to total fatty acid content in PC; the relative amount of 22:6n3 to total fatty acid content in CE; the relative amount of 22:6n3 to total fatty acid content in TG; the relative amount of 22:6n3 to total fatty acid content in total lipids; the relative amount of 18:0 to total fatty acid content in PC; the relative amount of 18:0 in total lipids to total fatty acid content; the relative amount of 18:2n6 to total fatty acid content in PC; the relative amount of 18:2n6 to total fatty acid content in CE; the relative amount of 18:2n6 to total fatty acid content in FA; the relative amount of 18:2n6 to total fatty acid content in TG; the relative amount of 18:2n6 to total fatty acid content in total lipids; the relative amount of 18:3n6 to total fatty acid content in PC; the relative amount of 18:3n6 to total fatty acid content in CE; the relative amount of 18:3n6 to total fatty acid content in TG; the relative amount of 18:3n6 to total fatty acid content in total lipids; the relative amount of 20:3n6 to total fatty acid content in PC; the relative amount of 20:3n6 to total fatty acid content in CE; and the relative amount of 20:3n6 to total fatty acid content in total lipids.

46. The method of claim 45, which further comprises measuring a second metabolite marker in the sample, wherein the second metabolite marker is selected from the group consisting of: the relative amount of 14:0 to total fatty acid content in TG; the relative amount of 14:0 to total fatty acid content in total lipids; the relative amount of 16:0 to total fatty acid content in PC; the relative amount of 16:0 to total fatty acid content in TG; the relative amount of 16:0 to total fatty acid content in total lipids; the relative amount of 16:1n7 to total fatty acid content in PC; the relative amount of 16:1n7 to total fatty acid content in CE; the relative amount of 16:1n7 to total fatty acid content in TG; the relative amount of 16:1n7 to total fatty acid content in FA; the relative amount of 16:1n7 to total fatty acid content in total lipids; the relative amount of 18:1n9 to total fatty acid content in PC; the relative amount of 18:1n9 to total fatty acid content in CE; the relative amount of 18:1n9 to total fatty acid content in total lipids; the relative amount of 20:3n9 to total fatty acid content in PC; the relative amount of 20:3n9 to total fatty acid content in CE; the relative amount of 20:3n9 to total fatty acid content in TG; the relative amount of 20:3n9 to total fatty acid content in total lipids; the relative amount of 20:3n6 to total fatty acid content in PC; the relative amount of 20:3n6 to total fatty acid content in CE; the relative amount of 20:3n6 to total fatty acid content in total lipids; the relative amount of 18:1n9 to total fatty acid content in FA; the relative amount of 22:6n3 to total fatty acid content in PC; the relative amount of 22:6n3 to total fatty acid content in CE; the relative amount of 22:6n3 to total fatty acid content in TG; the relative amount of 22:6n3 to total fatty acid content in total lipids; the relative amount of 18:0 to total fatty acid content in PC; the relative amount of 18:0 in total lipids to total fatty acid content; the relative amount of 18:2n6 to total fatty acid content in PC; the relative amount of 18:2n6 to total fatty acid content in CE; the relative amount of 18:2n6 to total fatty acid content in FA; the relative amount of 18:2n6 to total fatty acid content in TG; the relative amount of 18:2n6 to total fatty acid content in total lipids; the relative amount of 18:3n6 to total fatty acid content in PC; the relative amount of 18:3n6 to total fatty acid content in CE; the relative amount of 18:3n6 to total fatty acid content in TG; the relative amount of 18:3n6 to total fatty acid content in total lipids; the relative amount of 20:3n6 to total fatty acid content in PC; the relative amount of 20:3n6 to total fatty acid content in CE; and the relative amount of 20:3n6 to total fatty acid content in total lipids.

47. The method of claim 46, which further comprises measuring a third metabolite marker in the sample, wherein the third metabolite marker is selected from the group consisting of: the relative amount of 14:0 to total fatty acid content in TG; the relative amount of 14:0 to total fatty acid content in total lipids; the relative amount of 16:0 to total fatty acid content in PC; the relative amount of 16:0 to total fatty acid content in TG; the relative amount of 16:0 to total fatty acid content in total lipids; the relative amount of 16:1n7 to total fatty acid content in PC; the relative amount of 16:1n7 to total fatty acid content in CE; the relative amount of 16:1n7 to total fatty acid content in TG; the relative amount of 16:1n7 to total fatty acid content in FA; the relative amount of 16:1n7 to total fatty acid content in total lipids; the relative amount of 18:1n9 to total fatty acid content in PC; the relative amount of 18:1n9 to total fatty acid content in CE; the relative amount of 18:1n9 to total fatty acid content in total lipids; the relative amount of 20:3n9 to total fatty acid content in PC; the relative amount of 20:3n9 to total fatty acid content in CE; the relative amount of 20:3n9 to total fatty acid content in TG; the relative amount of 20:3n9 to total fatty acid content in total lipids; the relative amount of 20:3n6 to total fatty acid content in PC; the relative amount of 20:3n6 to total fatty acid content in CE; the relative amount of 20:3n6 to total fatty acid content in total lipids; the relative amount of 18:1n9 to total fatty acid content in FA; the relative amount of 22:6n3 to total fatty acid content in PC; the relative amount of 22:6n3 to total fatty acid content in CE; the relative amount of 22:6n3 to total fatty acid content in TG; the relative amount of 22:6n3 to total fatty acid content in total lipids; the relative amount of 18:0 to total fatty acid content in PC; the relative amount of 18:0 in total lipids to total fatty acid content; the relative amount of 18:2n6 to total fatty acid content in PC; the relative amount of 18:2n6 to total fatty acid content in CE; the relative amount of 18:2n6 to total fatty acid content in FA; the relative amount of 18:2n6 to total fatty acid content in TG; the relative amount of 18:2n6 to total fatty acid content in total lipids; the relative amount of 18:3n6 to total fatty acid content in PC; the relative amount of 18:3n6 to total fatty acid content in CE; the relative amount of 18:3n6 to total fatty acid content in TG; the relative amount of 18:3n6 to total fatty acid content in total lipids; the relative amount of 20:3n6 to total fatty acid content in PC; the relative amount of 20:3n6 to total fatty acid content in CE; and the relative amount of 20:3n6 to total fatty acid content in total lipids.

48-52. (canceled)

53. The method of claim 45, wherein the treatment of the diabetic condition comprises administration of a PPARs-gamma agonist, a PPARs-alpha agonist, and/or a PPARs-delta agonist.
